(19) United States
(12) Patent Application Publication
(10) Pub. No.: US 2008/0248068 A1
(43) Pub. Date: Oct. 9, 2008

(54) Use of Flagellin as an Adjuvant for Vaccine

(76) Inventors: Hans-Gustaf Ljunggren, Stockholm (SE); Steve Applequist, Stockholm (SE); Jorma Hinkula, Stockholm (SE); Bjorn Rozell, Stockholm (SE); Erik Rollman, Melbourne (AU)

Correspondence Address: YOUNG & THOMPSON, 209 Madison Street, Suite 500, ALEXANDRIA, VA, 22314, US

(21) Appl. No.: 11/579,324

(22) PCT Filed: May 2, 2005
(86) PCT No.: PCT/SE2005/000636

§ 371 Date: Jan. 19, 2007

Related U.S. Application Data
(60) Provisional application No. 60/521,501, filed on May 7, 2004.

(51) Int. Cl. A61K 39/00; A01K 67/027; C12N 15/00; C12N 15/11; C12N 5/06; A61P 37/00

(52) U.S. Cl. 424/282.1; 800/9; 435/320.1; 536/23.7; 435/325

(57) ABSTRACT

The present invention is directed to flagellin and its use as an adjuvant for vaccination. The invention can be used in vaccine formulations to improve immunity against any other antigen administered at the same localization. The antigen can be administered in the same construct as Flagellin or in any other formulation given at the same localization. As an alternative flagellin can be used to stimulate immunity against antigens expressed at a specific location. Flagellin can also be used to induce local inflammation with the purpose of creating a model for inflammation.

### BACKGROUND OF THE INVENTION

Delivery of naked DNA encoding antigens is able to induce adaptive immune responses'. This method has potential in it's ability to induce focused immune responses to defined antigens and benefits in it's ease of preparation and stability. However, improving the immunogenicity of DNA vaccination remains a fundamental goal considering the limited success in vaccinating humans and non-human primates using DNA alone²⁻¹⁵ compared to rodents. To date, these DNA vaccinations have proven ineffective unless combined with complex DNA-prime, protein/virus-boost regimes^{9,12,16}. To improve DNA-based vaccinations, vectors expressing cytokine/chemokine, and costimulatory genes have been used as “genetic adjuvants”¹⁷. The use of genetic adjuvants based on a single molecule have benefits in their ability to target the activation of specific immune cells but have limitations in that they may not effectively activate the immune system to the same degree as an infectious agent. Because of these limitations, there is a strong need to develop DNA encoded molecules that induce a more pleiotropic spectrum of local inflammatory responses when delivered using DNA vaccination methodology. Such molecules could be used in combination with a large variety of antigen-encoding DNA vaccines to induce strong adaptive immune responses without the need for mixed modality boosting regimes that need more care in preparation and storage than vaccines based on DNA alone.

Activation of the innate immune system through Toll-like receptors (TLR) is an effective-way to prime the immune system to activate strong adaptive immune responses. Once activated, TLR-expressing cells activate multiple arms of the immune system including anti-microbial effector molecules, type I and type II interferons, cytokines, chemokines, costimulatory molecules, and effective T and B cell priming by antigen presenting cells (APCs)¹⁸.

During vaccination, one would ideally like to have local production of short-lived inflammatory promoting molecules that activate TLRs. These in turn, could activate the innate immune system leading to the production of multiple factors and responses enhancing the responses to key antigens. Unfortunately, many TLR agonists are toxic¹⁹, the products of complex metabolic pathways specific to microbia (such as LPS or peptidoglycan), or cannot be produced by mammalian systems (such as unmethylated-CpG DNA motifs) and, are therefore not ideal for use as adjuvants in DNA-vaccines.

However, the observation that the polypeptide flagellin is an agonist for cells expressing TLR5²⁰ has opened up the possibility that eukaryotic cells may be able to produce this molecule. Phase-i flagellin from Salmonella (called FliC), the monomeric subunit protein which polymerizes to form the filaments of bacterial flagella, is a polypeptide without cystine residues²¹ and limited post-translational modification of lysine residues²². It has been extensively studied and the regions and residues of flagellin that interact with TLR5 have recently been defined²³. FliC is able to activate TLR5-dependent proinflammatory cyokine production and polymorphonuclear granulocyte recruitment in lung²⁴, intestinal epithelia^{25,26}, and is the major proinflammatory determinant of enteropathogenic Salmonella²⁷. It can activate mouse macrophages²⁸ and osteoblasts²⁹ to produce inflammatory mediators, human monocytes to produce TNFα³⁰ as well as induce human monocyte derived dendritic cells (DCs) to mature and upregulate costimulatory molecules³¹ and produce IFNγ, IL-10, IL-6, TNFα, and IL-12p70 but low IL-5 and IL-13³². These responses all demonstrate a bias to prime adaptive immunity towards a Th1-type response in vitro. FliC polypeptide produced and purified from the cytoplasm of transiently transfected mammalian cells also activates TLR5 expressing cells³³ suggesting that in mammalian cells FliC folds into an immunostimulatory form.

To develop more efficient ways of inducing local inflammation to be used in conjunction with DNA vaccination, we have constructed an expression vector that allows mammalian cells to express FliC on their surface. In vitro, cells transfected with these constructs were able to activate human monocytes to produce the inflammatory cytokines TNFα and upregulate CD80 and CD25 in a manner similar to LPS and recombinant flagellin isolated from bacteria. In vivo, mice given the FliC expressing vector in skin exhibited acute site-specific inflammatory responses and when combined with vectors expressing specific antigen, they developed marked increases in antigen-specific antibody responses. Surprisingly, we also observed cellular immunity to specific antigen suggesting that the FliC expressing vector induces a class of immune responses not normally seen in response to DNA encoded soluable antigens delivered intradermally.

### SUMMARY OF THE INVENTION

The present invention is directed to the use of Flagellin as a genetic adjuvant for vaccines. The invention consist of a nucleic acid construct encoding flagellin in a form that can be expressed either as membrane bound monomers or as soluble monomers. The flagellin adjuvant is administered at the same localization as a vaccine consisting of any substance capable of inducing specific immunity. The vaccine can be formulated as nucleic acids encoding genes expressed by pathogens or tumor cells or as proteins, peptides or attenuated pathogens or tumor cells. Alternatively, flagellin can be used to stimulate immunity against antigens expressed at a specific location. For example flagellin can be introduced into a tumor thereby inducing local inflammation resulting in activation of specific immunity against the tumor or in local toxicity. The gene for flagellin can be obtained from Salmonella typhimurion or any other organism expressing homologous genes.

By homologous we understand analogues or variants of the gene expressing the protein flagellin, in which one or more of the amino acid residues are replaced by different amino acid residues, or are deleted, or one or more amino acid residues are added to the original sequence of flagellin without changing considerably the activity of the resulting products as compared with wild type flagellin or its active fragments or fractions. The skilled person can test the activity as is done in the examples below.

In a preferred embodiment, any such analogue or variant has at least 40% identity or homology with the sequence of flagellin. More preferably, it has at least 50%, at least 60%, such as at least 65%, at least 70%, such as at least 75%, at least 80%, such as at least 85% or, most preferably, at least 90%, such as at least 95% identity or homology thereto.

### BRIEF DESCRIPTION OF THE DRAWING FIGURES

FIG. 1. Schematic representation of chimeric polypeptides, polypeptide expression, and reduction of FliC-Tm glycosylation by site directed mutagenesis (a).Primary structure of the predicted polypeptide encoded by thefliC-Tm ORF. κ designates the eukaryotic leader signal sequence for ER translocation, HA the HA-epitope, fliC the complete flagellin ORF, PDGF-Tm the platelet-derived growth factor receptor transmembrane domain. Recombinant fusion proteins detected in cytoplasmic cell lysates are shown. (b) Denaturing SDS-PAGE of cytoplasmic extracts from 293FT cells transfected with the indicated expression constructs (pcDNA3.1/Zeo(+)-based) detected using anti-HA-epitope antibodies or (c) anti-FliC antibodies. A culture supernatant from a vortexed overnight culture of S. typhimurium was used as a positive control for the anti-FliC antibody. (d) Detection of recombinant fusion proteins and their glycosylation in cytoplasmic extracts. Western blot demonstrating glycosylation of the unaltered version of FliC-Tm, deglycosylation of FliC-Tm using Endo H, and production of a reduced-glycosylated version of FliC-Tm (FliC-Tm(-gly)) after site-directed mutagenesis.

FIG. 2. Cells surface expression of FliC-Tm

Transiently transfected cells were subjected to flow cytometry using anti-HA epitope, anti-FliC, or isotype control antibodies (not shown). 293FT transfectants (anti-HA epitope or anti-FliC staining); pcDNA3.1/Zeocin(+)(Vector), filled histogram; pfliC-Tm, (-); pfliC-Tm (-gly) ( - - - ). Percentages of positive cells are indicated above the marker region. 293FT cells stained with anti-HA epitope antibodies are representative of 6 independent experiments and anti-FliC from 3 independent experiments.

FIG. 3. Activation of human monocytes by cells expressing FliC-Tm 293FT or HeLa cells were transfected as indicated. After two days supernatants and cells were collected and cells counted. Supernatants or living cells were mixed with resting monocytes, and 18 h later total cells and culture supernatants were harvested. Total cells were stained for CD80, and CD25; and supernatants were tested for the presence of TNFα. CD80, CD25 expression by monocytes incubated with 293FT cells (a), HeLa cells (b), or with LPS or recombinant FliC polypeptide at the indicated concentrations (c) were determined by flow cytometry. Monocytes were mixed with 293FT or HeLa cells transfected with pcDNA3.1/Zeo(+)(Vector) (filled histograms); pfliC-Tm (-), pfliC-Tm(-gly) ( - - - ). Percentages of positive cells are indicated above the marker region. Secreted TNFα expression by monocytes from cell-mixing experiments and stimulations were assayed for production by ELISA (d). 293FT and HeLa cell CD80 and CD25 data are representative of four independent experiments using independent PBMC donors. TNFα expression is representative of 2 independent experiments with both 293FT or HeLa cells. TNFα production was not seen in supernatants taken directly from cultures of plasmid transfected or mock transfected 293FT or HeLa cells (data not shown).

FIG. 4. FliC-Tm expression vectors induce acute, local inflammation

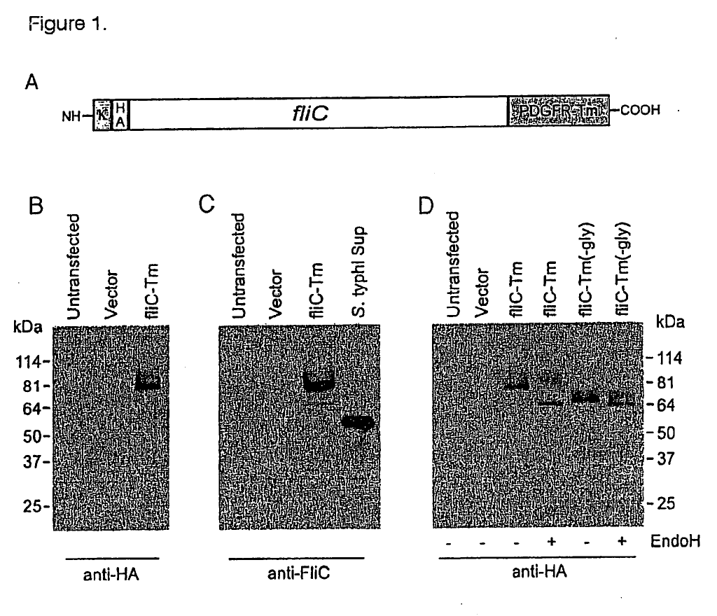

Gross morphology of the site of injection and histological analysis of the site after H&E staining are shown at days 0, 2 and 7 after one injection with the indicated DNA (0.5 μg each plasmid). Observations of the skin at, and immediately adjacent to the site of injection (a). Magnifications of the identical skin samples from the peritoneal muscle to the epithelial layer (b). Magnifications of identical sections focusing on changes in the upper dermis and epithelial layers (c). Smaller cropped sections from magnifications (d) representing shaded areas from identical sections in column (b). Data from days 1 and 3 are available online as Supplementary FIG. 1. Analyzed areas adjacent to sites of injection revealed no differences from normal skin (data not shown).

FIG. 5. FliC-Tm expression vectors potentiate DNA vaccination

(a) Immunization and sample isolation timeline. (b) Anti-OVA total IgG responses at day 61 after priming. (c) ELISPOT analysis of pooled peripheral blood T cell responses to MHC class I-restricted OVA peptide SIINFEKL at day 61. (d) Anti-OVA total IgG responses at day 74 after priming. (e) ELISPOT analysis of splenic T cell responses to SIINFEKL and whole OVA polypeptide. (f) Anti-OVA IgG1, (g) Ig2b, (h) IgG2c, and (i) IgA responses at day 74 after priming. IgA responses seen are from whole sera. The concentration of OVA-specific antibodies in serum samples from mice are expressed as the reciprocal of the last dilution of samples giving an optical density equal to, or higher than, the mean plus three standard deviations (IgG) or two standard deviations (IgA) (the determined cut off value for the assay) of the values of pre-immunization serum samples. Absorbance values equal to or above the cutoff value were considered positive. ELISPOT data is expressed as the calculated geometric mean of the antigen stimulated cells minus unstimulated cells. The cut-off for a given antigen was calculated as the group geometric mean of naïve animals plus two standard deviations. The * an ** represent significant difference of the response relative to pOVA immunizations without FliC-Tm expressing vectors, which are defined as P<0.05 and P<0.01, respectively.

FIG. 6 shows the vector map of pcDNA3.1/Zeo fliC-Tm(-gly)

Supplemental FIG. 1. The ORF of FliC Tm

atggagacagacacactcctgctatgggtactgctgctctgggttccagg
ttccactggtgactatccatatgatgttccagattatgctggggcccagc
cggccagaatggcacaagtcattaatacaaacagcctgtcgctgttgacc
cagaataacctgaacaaatcccagtccgctctgggcaccgctatcgagcg
tctgtcttccggtctgcgtatcaacagcgcgaaagacgatgcggcaggtc
aggcgattgctaaccgttttaccgcgaacatcaaaggtctgactcaggct
tcccgtaacgctaacgacggtatctccattgcgcagaccactgaaggcgc
gctgaacgaaatcaacaacaacctgcagcgtgtgcgtgaactggcggttc
agtctgctaacagcaccaactcccagtctgacctcgactccatccaggct
gaaatcacccagcgcctgaacgaaatcgaccgtgtatccggccagactca
gttcaacggcgtgaaagtcctggcgcaggacaacaccctgaccatccagg
ttggtgccaacgacggtgaaactatcgatatcgatctgaagcagatcaac
tctcagaccctgggtctggatacgctgaatgtgcaacaaaaatataaggt
cagcgatacggctgcaactgttacaggatatgccgatactacgattgctt
tagacaatagtacttttaaagcctcggctactggtcttggtggtactgac
cagaaaattgatggcgatttaaaatttgatgatacgactggaaaatatta
cgccaaagttaccgttacggggggaactggtaaagatggctattatgaag
tttccgttgataagacgaacggtgaggtgactcttgctggcggtgcgact
tccccgcttacaggtggactacctgcgacagcaactgaggatgtgaaaaa
tgtacaagttgcaaatgctgatttgacagaggctaaagccgcattgacag
cagcaggtgttaccggcacagcatctgttgttaagatgtcttatactgat
aataacggtaaaactattgatggtggtttagcagttaaggtaggcgatga
ttactattctgcaactcaaaataaagatggttccataagtattaatacta
cgaaatacactgcagatgacggtacatccaaaactgcactaaacaaactg
ggtggcgcagacggcaaaaccgaagttgtttctattggtggtaaaactta
cgctgcaagtaaagccgaaggtcacaactttaaagcacagcctgatctgg
cggaagcggctgctacaaccaccgaaaacccgctgcagaaaattgatgct
gctttggcacaggttgacacgttacgttctgacctgggtgcggtacagaa
ccgtttcaactccgctattaccaacctgggcaacaccgtaaacaacctga
cttctgcccgtagccgtatcgaagattccgactacgcgaccgaagtttcc
aacatgtctcgcgcgcagattctgcagcaggccggtacctccgttctggc
gcaggcgaaccaggttccgcaaaacgtcctctctttactgcgtgatccgc
ggctgcaggtcgacgaacaaaaactcatctcagaagaggatctgaatgct
gtgggccaggacacgcaggaggtcatcgtggtgccacactccttgccctt
taaggtggtggtgatctcagccatcctggccctggtggtgctcaccatca
tctcccttatcatcctcatcatgctttggcagaagaagccacgttag

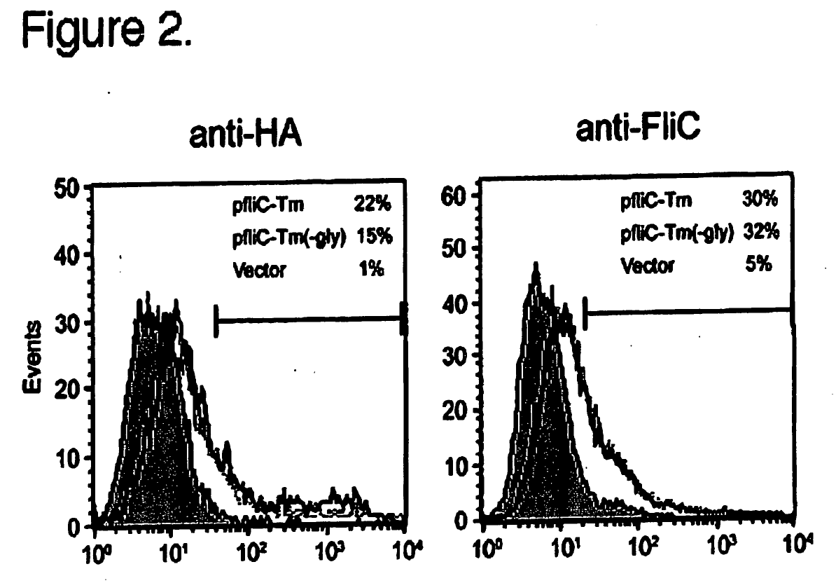

Supplemental FIG. 2. The ORF of FliC Tm(-gly)

atggagacagacacactcctgctatgggtactgctgctctgggttccagg
ttccactggtgactatccatatgatgttccagattatgctggggcccagc
cggccagatctatggcacaagtcattaatacaaacagcctgtcgctgttg
acccagaataacctggtcaaatcccagtccgctctgggcaccgctatcga
gcgtctgtcttccggtctgcgtatcaacagcgcgaaagacgatgcggcag
gtcaggcgattgctaaccgttttaccgcgaacatcaaaggtctgactcag
gcttcccgtaacgctaacgacggtatctccattgcgcagaccactgaagg
cgcgctgaacgaaatcaacaacaacctgcagcgtgtgcgtgaactggcgg
ttcagtctgctaccagcaccaactcccagtctgacctcgactccatccag
gctgaaatcacccagcgcctgaacgaaatcgaccgtgtatccggccagac
tcagttcaacggcgtgaaagtcctggcgcaggacaacaccctgaccatcc
aggttggtgccaacgacggtgaaactatcgatatcgatctgaagcagatc
aactctcagaccctgggtctggatacgctgaatgtgcaacaaaaatataa
ggtcagcgatacggctgcaactgttacaggatatgccgatactacgattg
ctttagacgatagtacttttaaagcctcggctactggtcttggtggtact
gaccagaaaattgatggcgatttaaaatttgatgatacgactggaaaata
ttacgccaaagttaccgttacggggggaactggtaaagatggctattatg
aagtttccgttgataagacgaacggtgaggtgactcttgctggcggtgcg
acttccccgcttacaggtggactacctgcgacagcaactgaggatgtgaa
aaatgtacaagttgcaaatgctgatttgacagaggctaaagccgcattga
cagcagcaggtgttaccggcacagcatctgttgttaagatgtcttatact
gataataacggtaaaactattgatggtggtttagcagttaaggtaggcga
tgattactattctgcaactcaaaataaagatggttccataagtattgata
ctacgaaatacactgcagatgacggtacatccaaaactgcactaaacaaa
ctgggtggcgcagacggcaaaaccgaagttgtttctattggtggtaaaac
ttacgctgcaagtaaagccgaaggtcacaactttaaagcacagcctgatc
tggcggaagcggctgctacaaccaccgaaaacccgctgcagaaaattgat
gctgctttggcacaggttgacacgttacgttctgacctgggtgcggtaca
gaaccgtttcaactccgctattaccaacctgggcaacaccgtaaacaacc
tgaattctgcccgtagccgtatcgaagattccgactacgcgaccgaagtt
tccaacatgtctaaagcgcagattctgcagcaggccggtacctccgttct
ggcgcaggcgaaccaggttccgcaaaacgtcctctctttactgcgagcag
taccccgggatccgcggctgcaggtcgacgaacaaaaactcatctcagaa
gaggatctgaatgctgtgggccaggacacgcaggaggtcatcgtggtgcc
acactccttgccctttaaggtggtggtgatctcagccatcctggccctgg
tggtgctcaccatcatctcccttatcatcctcatcatgctttggcagaag
aagccacgttag

### DETAILED DESCRIPTION OF THE INVENTION

### Construction of Chimeric Polypeptide's

To express FliC on the surface of mammalian cells, we constructed vectors containing theflic gene from S. typhimurium in the mammalian expression vector pDisplay (pDisp/fliC-Tm). The coding region of the PCR product was identical to the DNA sequence of S. typhimurium phase-I flagellin, and the naturally occurring stop codon was changed to allow ribosomal read-through into the region of the vector containing the human PDGFR transmembrane domain. A fragment containing the complete open reading frame was excised and transferred to the expression vector pcDNA3.1/Zeo(+) for use in further experiments. See Supplemental informnation FIGS. 1 and 2 for the key ORFs described here. See the section “Sequences” for detailed information. 293FT cells were transiently transfected with the pcDNA3.1/fliC-Tm (pfliC-Tm) expression vector, and cell lysates were analyzed by Western blotting. We expected a mature polypeptide product of ≅61 kDa. A schematic representation of the primary structure of the expected pre-processed polypeptide and expression of the mature polypeptide is shown in FIGS. 1a and 1b, respectively. Proteins of identical molecular weight were detected using both anti-HA tag (FIG. 1b) and anti-FliC antibodies (FIG. 1c) but with larger than expected size at ≧7 kDa and a more disperse band at ≅3 kDa.

The structure of native flagellin isolated from S. typhimurium has been well characterized²¹ and is not glycosylated. However, multiple eukaryotic N-linked glycosylation sites were identified in the coding sequence offlic. It was thus possible that the larger molecular weight products of FliC-Tm produced by 293FT cells could be due to N-linked glycosylation. To address this, whole cytoplasmic cell lysates of pfliC-Tm transfected 293FT cells were treated with Endo H which removes simple carbohydrate structures (high-mannose and hybrid found in the ER) but not complex carbohydrate structures (found upon Golgi processing). Upon Endo H treatment, the ≅77 kDa FliC-Tm migrated at the expected size of ≅61 kDa while the running properties of the larger ≅83 kDa polypeptide did not change (FIG. 1d).

To prevent N-linked glycosylation of these residues of FliC in mammalian cells, the coding sequence of fliC was changed. Either Asn itself was altered or signal sequences needed for glycosylation attachment to Asn. Changes were chosen by identifying predicted amino acid residues present at similar locations within flagellin molecules of other flagellated bacteria (Table I).

TABLE I
Summary of amino acid changes made to eliminate ASN-linked
glycosylation signal sequences

| Predicted | Predicted | | |
|---|---|---|---|
| Asn-linked | Asn-linked | | Organism and GenBank |
| glycosylation | glycosylation | AA | accession number on which the |
| site | sequence | change | sequence change is based |
| 19* | NKSQ | 19 N/V | H. felis #Y11602 |
| 101 | NSTN | 101 | E. coli #AF169323 |
| 200 | NSTF | 200 | S. choleraesuis #AF159459 |
| 346 | NTTK | 346 | S. typhimurium #M11332 |
| 446 | NLTS | 448 | S. enterica #U06206 |
| 465 | NMSR | 468 | S. enterica #U06205 |

*amino acid position

It was expected that changing these residues would lead to the production of a polypeptide that would migrate at the expected size of ≅61 kDa but still fold into an immunostimulatory form. The nucleotide sequence of fliC-Tm was changed by site directed mutagenesis, and the resulting construct was called pfliC-Tm(-gly). Polypeptides produced by 293FT cells transfected with pfliC-Tm(-gly) were of ≅66/69 kDa respectively (FIG. 1d). These results highlight a possible difficulty in the eukaryotic production of desired polypeptides from microorganisms.

### Cell Surface Expression of FliC

To determine if cells transfected with the expression constructs expressed the FliC-Tm polypeptide at their surface, cells were stained with anti-FliC and anti-HA antibodies followed by FACS® analysis. 293FT cell cultures transfected with either pfliC-Tm or pfliC-Tm(-gly) contained cells detectable with an anti-HA epitope antibody (FIG. 2) but not with an isotype-control antibody (data not shown). Mock transfected cells (data not shown) or cells transfected with an empty vector (FIG. 2) gave background staining. Cells transfected with pfliC-Tm or pfliC-Tm(-gly) also expressed proteins detectable by anti-FliC antibodies (FIG. 2). Similar or greater percentages of cells staining positive for pfliC-Tm or pfliC-Tm(-gly) were detected using the polyclonal anti-FliC antibody. HeLa cells were also transfected with pfliC-Tm, pfliC-Tm(-gly), or empty vector and similar surface expression was observed using anti-HA and anti-FliC antibodies.

### Activation of Human Monocytes by Cells Expressing FliC

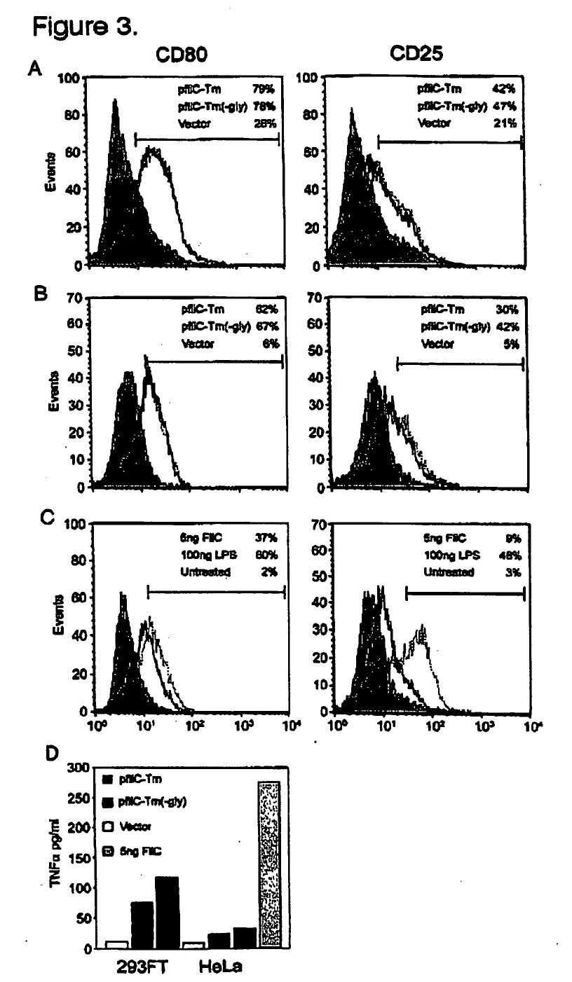

Adherence-enriched human PBMCs (monocytes) produce inflammatory factors in response to recombinant S. typhimurium flagellin³⁰. To assess whether human cells expressing FliC-Tm on their surface are able to activate human monocytes, we incubated pfliC-Tm or pfliC-Tm(-gly) transfected 293FT cells with resting monocytes. Cells were transfected with the indicated vectors and surface expression of FliC-Tm or FliC-Tm(-gly) was analyzed. Total cultures of transfected cells were washed with PBS then mixed with monocytes, incubated for 18 h, and analyzed for TNFα production and changes in surface expression of CD80 and CD25. Cultures of 293FT cells expressing FliC-Tm or FliC-Tm(-gly) were able to induce monocytes to upregulate cell surface expression of CD80 and CD25 compared to controls (FIG. 3a). The changes induced. were similar to those seen after treatment with LPS or recombinant FliC polypeptide (FIG. 3c). FliC-Tm or fliC-Tm(-gly) expressing cells were also able to induce production of TNFα (FIG. 3d). Furthermore, supernatants from cultures of transfected 293FT cells were also able to upregulate CD80 and CD25 levels on monocytes (data not shown). NF-κB activation in response to Salmonella-derived FliC (indicative of TLR activation) has been reported to occur in 293 but not in HeLa cells³⁶ raising the possibility that transfection of 293FT cells with FliC-Tm expressing constructs leads to the production of undefined factors from 293FT cells that are able to activate monocytes. To test this hypothesis, transfection and cell mixing experiments were also performed using HeLa cells transfected with pfliC-Tm or pfliC-Tm(-gly). These cells, mixed with monocytes, were also able to induce monocyte activation similar to 293FT expressing FliC-Tm or FliC-Tm(-gly) but not cells transfected with the empty vector (FIGS. 3b, d). Supernatants from cultures of transfected HeLa cells also activated monocytes (data not shown). 293FT and HeLa cells were negative for staining by anti-CD80, anti-CD25 and anti-HLA-DR (data not shown).

The combination of our finding that membrane bound flagellin induces activation of monocytes with the activation of monocytes by soluble flagellin shows that it will be possible to induce activation by cells transfected with a vector expressing soluble flagellin.

### Flagellin Expressing Vectors Induce Local, Acute Inflammation

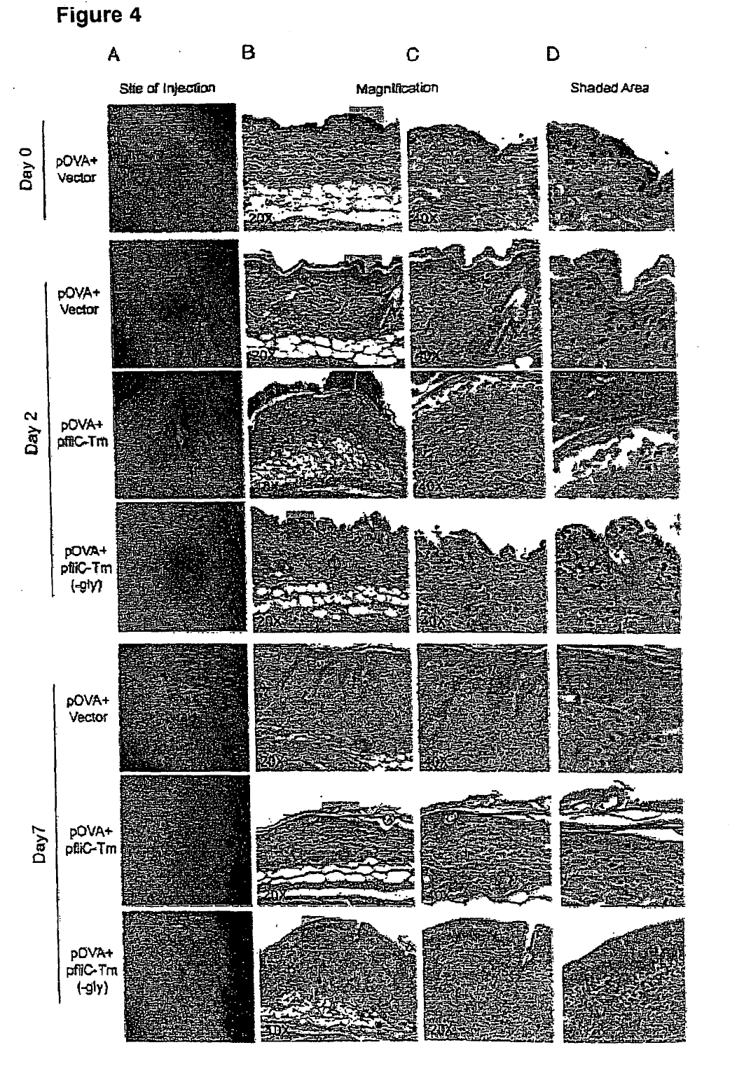

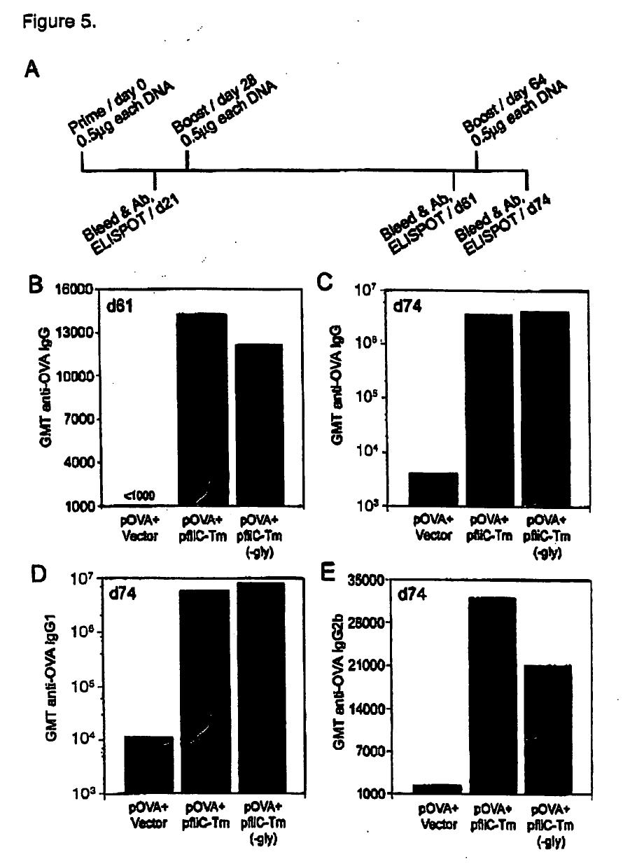

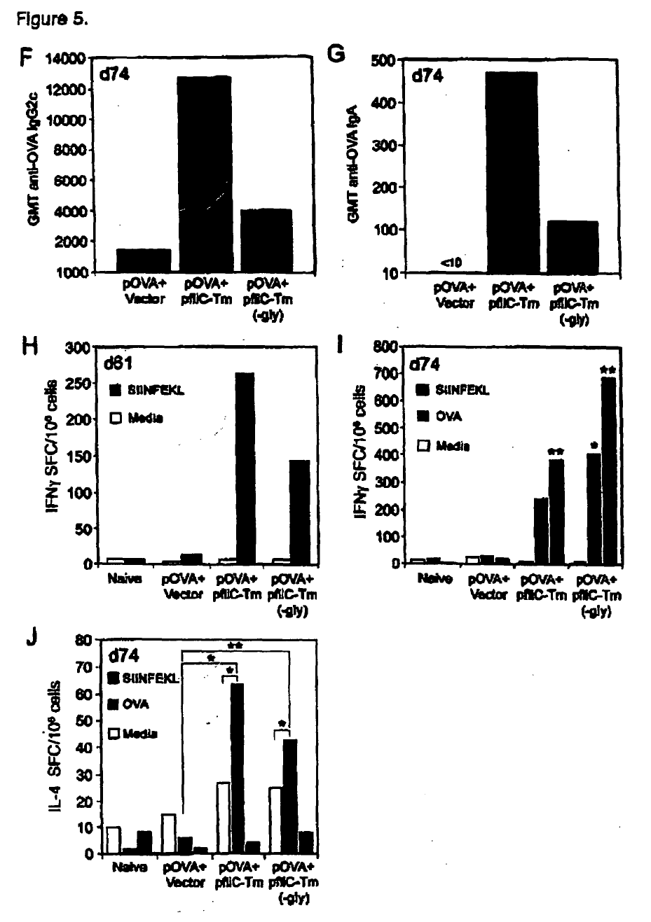

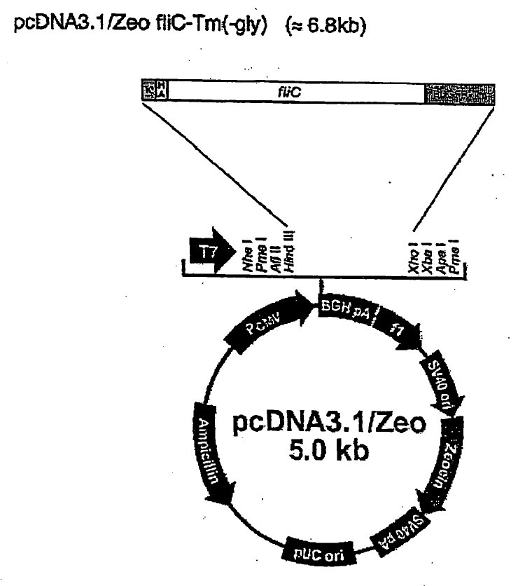

determine if FliC-Tm expressing vectors are capable of inducing an inflammatory response in vivo, we used the gene-gun method to inject pfliC-Tm or pfliC-Tm(-gly) plasmids into mice. Gold beads were coated with a test vector containing chicken ovalbumin (pOVA) together with an empty expression vector (pcDNA3.1/Zeo(+)) called Vector or in combination with pfliC-Tm or pfliC-Tm(-gly). Mice were immunized, and each site of injection was photographed immediately after sacrifice at the indicated days (FIG. 4). The injection site together with surrounding skin was dissected, fixed, and subjected to histological analysis to determine if there were differences in local responses between mice vaccinated with different DNA preparations.

Gross morphology of the injection sites revealed clear differences in the type of responses elicited relative to the type of plasmid delivered (FIG. 4a). Mice injected with the plasmid pOVA+Vector showed a slight local reaction two days post-injection, characterized primarily by a yellowish-brown tinge likely due to deposition of the gold particles. In contrast, mice injected with pOVA+pfliC-Tm or pOVA+pfliC-Tm(-gly) developed severe, but local tissue reactions characterized by swelling and central ulceration of the injection site. Seven days post-injection, the skin was grossly normal in all groups of mice.

Histological analysis of the site of injection, revealed similarities, but also striking differences between mice injected with pOVA+Vector, compared to mice injected with pOVA+pfliC-Tm or pOVA+pfliC-Tm(-gly). In mice sacrificed directly post injection, the distribution of gold particles were found in the epidermis and subepidermal dermis (FIGS. 4b,c,d). On days one and two post injection (FIGS. 4b-d), mice given pOVA+Vector, the developed epidermal hyperplasia, subcomeal pustule formation, increased cellular density in the dermis with infiltration of neutrophillic granulocytes (NG), and an inflammatory reaction extending to, but not involving, the hypodermal fat. On day three, the inflammation was resolving, while the superficial necrotic epidermal layers and pustules, were detaching from the site of injection. In contrast, injection of pOVA combined with either of the FliC-Tm expressing plasmids, led to a more rapid and severe inflammatory reaction, involving also the hypodermis, extending to and involving the superficial part of the panniculus muscle. On day one, epidermal necrosis was observed in the central injection site and the denuded area covered with fibrin was densely infiltrated by granulocytes. In the dermis and the hypodermis, the inflammatory reaction led to the development of a panniculitis, with dense infiltrates of neutrophilic granulocytes. At day two (FIGS. 4b-d), similar observations were made but in addition there was an increased hyperemia, with marginating neutrophils in the vasculature. At day three, the lateral parts of the injection site displayed epidermal hyperplasia, while the central region still was characterized epidermal pustule formation; the acute inflammatory reaction in the dermal parts persisted, albeit somewhat reduced. The hyperemic vessels were less prominent, and there was evidence of an early wound healing reaction. In all animals tested, there was an aggregation of gold particles in the detaching epidermal region and bead aggregation was seen in the dermis. By day seven (FIG. 4), there was still evidence of epidermal hyperplasia in all groups, with hypergranulosis, and hyperkeratosis, but the inflammation reaction had mostly resolved and many of the remaining gold beads were found clumped together in dermal aggregates. Scar formation was seen in the central injection site, but not in the lateral parts.

The inflammatory responses observed in this study appear to be unique to the use of FliC-Tm as similar responses have not been observed or reported with the use of any other genetic encoded adjuvants such as IL-2, IL-12, or GM-CSF (our own observations)¹⁷. We observed a direct correlation between the presence of the plasmid coated gold beads and inflammation, suggesting that the deposition of the DNA cargo was responsible for changes in the mice. This correlation also extends to the presence of inflammation and enhancement of immune responses to antigen (mentioned below).

Similar results would be expected following gene-gun immunizations using a vector encoding soluble flagellin. Our results show that flagellin can be used as an adjuvant for nucleic acid based vaccinations. Flagellin can be expressed wither as a membrane bound molecule or as a soluble molecule. In both situations, local inflammation will be induced which results in enhanced immune responses against antigens expressed in the tissue.

### Flagellin Expressing Vectors Potentiate DNA Vaccination

To determine if FliC-Tm expressing vectors could enhance adaptive immune responses to DNA encoded soluble antigen (OVA) we used the gene-gun method to vaccinate mice. Mice were immunized with pOVA+Vector, pOVA+pfliC-Tm, or pOVA+fliC-Tm(-gly) according to the immunization schedule illustrated in FIG. 5a. Blood was taken at the indicated days, and serum was tested for the presence of anti-OVA antibodies. Anti-OVA IgG responses were undetectable at day 21 (data not shown). At day 61 anti-OVA IgG levels were measured, and after the final boost, anti-OVA IgG, IgG-isotypes, and IgA were measured (FIGS. 5d, f-i). After one boost, increases in anti-OVA total IgG responses in pfliC-Tm and pfliC-Tm(-gly) vaccinated mice were seen but not in mice given pOVA+Vector (FIG. 5b). After a second boost, higher anti-OVA total IgG titers were observed, including increases in anti-OVA IgG-isotypes IgG1 (FIG. 5f), IgG2b (FIG. 5g), and IgG2c (FIG. 5h), as well as IgA (FIG. 5i). Corresponding antibody responses were slight or undetectable in mice receiving pOVA+Vector alone.

Lymphocytes were then tested for the presence of antigen-specific T cells in peripheral blood at days 21 and 61, and in the spleens of mice at day 74. ELISPOT analysis of PBMCs at day 21 failed to detect antigen-specific T cell responses in any groups (data not shown). However, analysis of blood at day 61 revealed the presence of circulating IFNγ-producing T cells responding to the H-2K^{b}-restricted OVA peptide SIINFEKL (residues 257-264) in mice that had received pOVA+pfliC-Tm or pOVA+pfliC-Tm(-gly) but not in mice that had received pOVA+Vector (FIG. 5c). Spleens from mice at day 74 were tested for the presence of IFNγ-producing T cells able to respond to SIINFEKL as well as whole OVA (FIG. 5e). Levels of IFNγ detected in response to SIINFEKL and whole OVA were significantly higher in mice vaccinated with pOVA+pfliC-Tm or pOVA+pfliC-Tm(-gly) than in mice receiving pOVA+Vector alone. T cells from these mice (pOVA+Vector) remained unresponsive to either peptide or polypeptide relative to control peptide or protein.

Gene-gun challenge of naive mice with plasmids encoding soluble antigens (such as pOVA) results primarily in a Th2-like response dominated by antibody production^{37,38}. However, analysis of the immune responses induced by the addition of FliC-Tm expressing vectors in combination with pOVA revealed a number of interesting observations. When pfliC-Tm vectors were used as adjuvants, anti-OVA antibodies appeared after only one DNA boost immunization while no anti-OVA responses were seen in the group given pOVA+Vector. We believe that the undetectable anti-OVA IgG responses in mice given pOVA+Vector are likely due to the low amount of plasmid used during immunization (0.5 μg) and lack of significant boosting. However, the appearance anti-OVA IgG responses after a second boost in the pOVA+Vector group indicates that the mice were receiving the pOVA plasmid. The three-log higher titers of anti-OVA IgG and increases in IgG isotypes when pfliC-Tm plasmids were included demonstrates its ability to act as an adjuvant. The appearance of IgA in the sera is also a promising sign suggesting that FliC-Tm expressing plasmids could be a useful addition to DNA vaccinations wishing to elicit mucosal antibody defences.

Surprisingly, when FliC-Tm expressing plasmids were included in our vaccinations we were able to induce cellular immune responses to an antigen that has been shown to elicit only antibody responses³⁸. It has been suggested that levels of secreted OVA may be too low to load the MHC class I presentation pathway in order to elicit CTL responses after gene gun vaccination³⁸. Here however, we have vaccinated with less plasmid and fewer injections compared to other gene gun studies but were able to elicit antigen-specific MHC class I-dependent T cell responses when FliC-Tm expressing vectors were used.

The gene gun delivery of DNA has been shown to induce a Th2-promoting signal that is dominant to immunostimulatory CpG motifs in DNA vaccines³⁷. Therefore, it is unlikely that the 13 CpG motifs found in the FliC-Tm ORF (data not shown), of which only one is optimal³⁹, contributes to the T cell responses seen in this study. It may be more likely that the spectrum of inflammatory factors FliC can induce in vitro (Th1)³² is able to “license” local or recruited APCs in vivo to initiate CD8⁺ T cell responses against secreted OVA. Indeed, cross-priming has been seen in other in vivo systems studying the effects of bacterial products⁴⁰⁻⁴³ and viral infection⁴⁴ to induce CD8⁺ T cell responses to extracellular antigens. Regardless of the molecular mechanisms involved, the delivery of FliC-Tm expressing plasmids by gene gun-vaccination not only elicits significant increases in antibody responses to DNA encoded antigens after fewer immunizations compared to those in controls but also increases the breadth of the response to induce MHC class I-restricted cellular immunity. These results suggest that FliC-Tm induces Th1-like responses in vivo and that the use of FliC-Tm expressing vectors in combination with key pathogen antigens could induce successful protective vaccination.

Its interesting to speculate on the fate of FliC-Tm polypeptide and the cells which express it. FliC-Tm may be cleaved from the surface of cells by serine proteinases produced by the neutrophil infiltrate in the skin of injected mice⁴⁵. Alternatively, cells expressing FliC-Tm could be eliminated by the stressful effects of the local inflammation they induce or possibly by TLR5 expressing phagocytic APC. In either case, the passive or active elimination of the pfliC-Tm vector or cells expressing FliC-Tm would result in the resolution of the inflammatory process, an observation which we have seen here. It also would seem to be a key step in avoiding possible chronic inflammation.

Bacteria expressing flagella containing antigenic inserts have been used in experimental systems as vaccines⁴⁶, and recombinant flagellin has also been used as an adjuvant in combination with peptide antigens to induce CD4 T cell responses³³. Currently, however, the practical use of these approaches are limited by the heterogenous nature of molecular preparations, either by possible contamination, or by the presence of the complete attenuated bacteria. The use of a DNA expression vector that enables mammalian cells to express flagellin at their surface has distinct advantages for vaccination efficacy such as ease of preparation, ability to remove contamination with unwanted inflammation promoting molecules, and stability.

Flagellin can be used as an adjuvant together with any antigen that induces immune responses. Examples of such antigens are DNA or RNA encoding antigens from tumors or pathogens, proteins, complete pathogens such as viral particles, bacteria, parasites, tumor cells or cells infected with intracellular pathogens. Flagellin can also be introduced into tissues or cells expressing antigens against which immunity should be generated. Examples of such tissues are tumors or sites for infection. As an alternative flagellin can be used to induce local inflammation resulting in toxicity against cells located at the inoculation site. This approach would be of particular use against tumors and possibly against autoimmune diseases.

The ability of flagellin to induce local inflammation can also be used to create an animanl model for inflammation or chronic inflammation. This is done by introduction of flagellin under a tissue specific promoter into a transgenic animal. The use of an inducible promoter would have several advantages. The transgenic animal can be used for studies of inflammation including the studies of anti-inflammatory drugs, inhibitors of inflammatory pathways or to study mechanisms involved in inflammation.

The use of a membrane bound flagellin monomer provides several advantages for example it limits the inflammatory response to the tissue where flagellin is expressed thereby limiting the risk for adverse effects such as systemic inflammatory responses, tissue damage in other tissues which in turn can potentially result in for example autoimmunity. Expression of membrane bound flagellin also increases the possibility of targeting the inflammatory response to a specific tissue such as a tumor or any tissue expressing a gene to which immunity is required. It may also reduce the risk for over stimulating the immune system which may result in tolerance development, inadequate immune responses or even toxic effects.

Flagellin may be administrated in a gene gun composition comprising a dose of at least 0,5 μg, e.g. 0,5-10 μg, preferably 0,5-5 μg of flagellin plasmid nucleic acid as adjuvant together with approximately the same dose of plasmid antigen nucleic acid. The adjuvant and the antigen nucleic acid may be administrated in separate compositions or together in the same composition in different or the same plasmid. The dose may be administrated 1 to 3 times a day.”

### Sequences

Accession numbers used here were-GenBank # D13689 and Swiss-Prot link P06179 for S. typhimurium phase-I flagellin° FliC.
First the ORF of FliC Tm followed by FliC Tm(-gly).
FliC Tm Predicted complete neuclotide and amino acid sequence fromfliC (S. typhimurium; GenBank accession number D13689) as a genetic fusion with the Leader, HA-tag, myc-tag, and PDGFR transmembrane sequence found in the commerical vector pDisplay (Invitrogen, Carlsbad, Calif., U.S.A.).

```
atggagacagacacactcctgctatgggtactgctgctctgggttccaggttccactggt
  M E T D T L L L W V L L L W V P G S T G
gactatccatatgatgttccagattatgctggggcccagccggccagaatggcacaagtc
  D Y P Y D V P D Y A G A Q P A R M A Q V
attaatacaaacagcctgtcgctgttgacccagaataacctgaacaaatcccagtccgct
  I N T N S L S L L T Q N N L N K S Q S A
ctgggcaccgctatcgagcgtctgtcttccggtctgcgtatcaacagcgcgaaagacgat
  L G T A I E R L S S G L R I N S A K D D
gcggcaggtcaggcgattgctaaccgttttaccgcgaacatcaaaggtctgactcaggct
  A A G Q A I A N R F T A N I K G L T Q A
tcccgtaacgctaacgacggtatctccattgcgcagaccactgaaggcgcgctgaacgaa
  S R N A N D G I S I A Q T T E G A L N E
atcaacaacaacctgcagcgtgtgcgtgaactggcggttcagtctgctaacagcaccaac
  I N N N L Q R V R E L A V Q S A N S T N
tcccagtctgacctcgactccatccaggctgaaatcacccagcgcctgaacgaaatcgac
  S Q S D L D S I Q A E I T Q R L N E I D
cgtgtatccggccagactcagttcaacggcgtgaaagtcctggcgcaggacaacaccctg
  R V S G Q T Q F N G V K V L A Q D N T L
accatccaggttggtgccaacgacggtgaaactatcgatatcgatctgaagcagatcaac
  T I Q V G A N D G E T I D I D L K Q I N
tctcagaccctgggtctggatacgctgaatgtgcaacaaaaatataaggtcagcgatacg
  S Q T L G L D T L N V Q Q K Y K V S D T
gctgcaactgttacaggatatgccgatactacgattgctttagacaatagtacttttaaa
  A A T V T G Y A D T T I A L D N S T F K
gcctcggctactggtcttggtggtactgaccagaaaattgatggcgatttaaaatttgat
  A S A T G L G G T D Q K I D G D L K F D
gatacgactggaaaatattacgccaaagttaccgttacggggggaactggtaaagatggc
  D T T G K Y Y A K V T V T G G T G K D G
tattatgaagtttccgttgataagacgaacggtgaggtgactcttgctggcggtgcgact
  Y Y E V S V D K T N G E V T L A G G A T
tccccgcttacaggtggactacctgcgacagcaactgaggatgtgaaaaatgtacaagtt
  S P L T G G L P A T A T E D V K N V Q V
gcaaatgctgatttgacagaggctaaagccgcattgacagcagcaggtgttaccggcaca
  A N A D L T E A K A A L T A A G V T G T
gcatctgttgttaagatgtcttatactgataataacggtaaaactattgatggtggttta
  A S V V K M S Y T D N N G K T I D G G L
gcagttaaggtaggcgatgattactattctgcaactcaaaataaagatggttccataagt
  A V K V G D D Y Y S A T Q N K D G S I S
attaatactacgaaatacactgcagatgacggtacatccaaaactgcactaaacaaactg
  I N T T K Y T A D D G T S K T A L N K L
ggtggcgcagacggcaaaaccgaagttgtttctattggtggtaaaacttacgctgcaagt
  G G A D G K T E V V S I G G K T Y A A S
aaagccgaaggtcacaactttaaagcacagcctgatctggcggaagcggctgctacaacc
  K A E G H N F K A Q P D L A E A A A T T
accgaaaacccgctgcagaaaattgatgctgctttggcacaggttgacacgttacgttct
  T E N P L Q K I D A A L A Q V D T L R S
gacctgggtgcggtacagaaccgtttcaactccgctattaccaacctgggcaacaccgta
  D L G A V Q N R F N S A I T N L G N T V
aacaacctgacttctgcccgtagccgtatcgaagattccgactacgcgaccgaagtttcc
  N N L T S A R S R I E D S D Y A T E V S
aacatgtctcgcgcgcagattctgcagcaggccggtacctccgttctggcgcaggcgaac
  N M S R A Q I L Q Q A G T S V L A Q A N
caggttccgcaaaacgtcctctctttactgcgtgatccgcggctgcaggtcgacgaacaa
  Q V P Q N V L S L L R D P R L Q V D E Q
aaactcatctcagaagaggatctgaatgctgtgggccaggacacgcaggaggtcatcgtg
  K L I S E E D L N A V G Q D T Q E V I V
gtgccacactccttgccctttaaggtggtggtgatctcagccatcctggccctggtggtg
  V P H S L P F K V V V I S A I L A L V V
ctcaccatcatctcccttatcatcctcatcatgctttggcagaagaagccacgttaggcg
  L T I I S L I I L I M L W Q K K P R - A
gccgctcgag
  A A R
```

Predicted polypeptide defined as functional domains.

METDTLLLWV LLLWVPGSTG DYPYDVPDYA GAQPARMAQV
INTNSLSLLT QNNLNKSQSA LGTAIERLSS GLRINSAKDD
AAGQAIANRF TANIKGLTQA SRNANDGISI AQTTEGALNE
INNNLQRVRE LAVQSANSTN SQSDLDSIQA EITQRLNEID
RVSGQTQFNG VKVLAQDNTL TIQVGANDGE TIDIDLKQIN
SQTLGLDTLN VQQKYKVSDT AATVTGYADT TIALDNSTFK
ASATGLGGTD QKIDGDLKFD DTTGKYYAKV TVTGGTGKDG
YYEVSVDKTN GEVTLAGGAT SPLTGGLPAT ATEDVKNVQV
ANADLTEAKA ALTAAGVTGT ASVVKMSYTD NNGKTIDGGL
AVKVGDDYYS ATQNKDGSIS INTTKYTADD GTSKTALNKL
GGADGKTEVV SIGGKTYAAS KAEGHNFKAQ PDLAEAAATT
TENPLQKIDA ALAQVDTLRS DLGAVQNRFN SAITNLGNTV
NNLTSARSRI EDSDYATEVS NMSRAQILQQ AGTSVLAQAN
QVPQNVLSLL R AVP DPRLQVDEQ KLISEEDLNA VGQDTQEVIV
VPHSLPFKVV VISAILALVV LTIISLIILI MLWQKKPR

Underlined regions: Regions encoded by pDisplay

METDTLLLWV LLLWVPGSTG DYPYDVPDYA GAQPAR PRLQVDEQ
KLISEEDLNA VGQDTQEVIV VPHSLPFKVV VISAILALVV
LTIISLIILI MLWQKKPR

Grey Shaded area: IgK-leader sequence

METDTLLLWV LLLWVPGSTG D

Magenta Shaded area: HA-tag

YPYDVPDYA

Blue Shaded area: fliC

MAQV INTNSLSLLT QNNLNKSQSA LGTAIERLSS GLRINSAKDD
AAGQAIANRF TANIKGLTQA SRNANDGISI AQTTEGALNE
INNNLQRVRE LAVQSANSTN SQSDLDSIQA EITQRLNEID
RVSGQTQFNG VKVLAQDNTL TIQVGANDGE TIDIDLKQIN
SQTLGLDTLN VQQKYKVSDT AATVTGYADT TIALDNSTFK
ASATGLGGTD QKIDGDLKFD DTTGKYYAKV TVTGGTGKDG
YYEVSVDKTN GEVTLAGGAT SPLTGGLPAT ATEDVKNVQV
ANADLTEAKA ALTAAGVTGT ASVVKMSYTD NNGKTIDGGL
AVKVGDDYYS ATQNKDGSIS INTTKYTADD GTSKTALNKL
GGADGKTEVV SIGGKTYAAS KAEGHNFKAQ PDLAEAAATT
TENPLQKIDA ALAQVDTLRS DLGAVQNRFN SAITNLGNTV
NNLTSARSRI EDSDYATEVS NMSRAQILQQ AGTSVLAQAN
QVPQNVLSLL R

Green Shaded area: PDGFR-transmembrane domain

A VGQDTQEVIV VPHSLPFKVV VISAILALVV LTIISLIILI
MLWQKKPR

FliC Tm (-gly) Predicted complete neuclotide and amino acid sequence from fliC (S. typhimurium; GenBank accession number D13689) as a genetic fusion with the Leader, HA-tag, myc-tag, and PDGFR transmembrane sequence found in the commerical vector pDisplay (Invitrogen, Carlsbad, Calif., U.S.A.). The fliC ORF has been altered to result in 6 predicted amino acid differences from D13689.

```
atggag
  M E
acagacacactcctgctatgggtactgctgctctgggttccaggttccactggtgactat
  T D T L L L W V L L L W V P G S T G D Y
ccatatgatgttccagattatgctggggcccagccggccagatctatggcacaagtcatt
  P Y D V P D Y A G A Q P A R S M A Q V I
aatacaaacagcctgtcgctgttgacccagaataacctggtcaaatcccagtccgctctg
  N T N S L S L L T Q N N L V K S Q S A L
ggcaccgctatcgagcgtctgtcttccggtctgcgtatcaacagcgcgaaagacgatgcg
  G T A I E R L S S G L R I N S A K D D A
gcaggtcaggcgattgctaaccgttttaccgcgaacatcaaaggtctgactcaggcttcc
  A G Q A I A N R F T A N I K G L T Q A S
cgtaacgctaacgacggtatctccattgcgcagaccactgaaggcgcgctgaac
  R N A N D G I S I A Q T T E G A L N
gaaatcaacaacaacctgcagcgtgtgcgtgaactggcggttcagtctgctaccagcacc
  E I N N N L Q R V R E L A V Q S A T S T
aactcccagtctgacctcgactccatccaggctgaaatcacccagcgcctgaacgaaatc
  N S Q S D L D S I Q A E I T Q R L N E I
gaccgtgtatccggccagactcagttcaacggcgtgaaagtcctggcgcaggacaacacc
  D R V S G Q T Q F N G V K V L A Q D N T
ctgaccatccaggttggtgccaacgacggtgaaactatcgatatcgatctgaagcagatc
  L T I Q V G A N D G E T I D I D L K Q I
aactctcagaccctgggtctggatacgctgaatgtgcaacaaaaatataaggtcagcgat
  N S Q T L G L D T L N V Q Q K Y K V S D
acggctgcaactgttacaggatatgccgatactacgattgctttagacgatagtactttt
  T A A T V T G Y A D T T I A L D D S T F
aaagcctcggctactggtcttggtggtactgaccagaaaattgatggcgatttaaaattt
  K A S A T G L G G T D Q K I D G D L K F
gatgatacgactggaaaatattacgccaaagttaccgttacggggggaactggtaaagat
  D D T T G K Y Y A K V T V T G G T G K D
ggctattatgaagtttccgttgataagacgaacggtgaggtgactcttgctggcggtgcg
  G Y Y E V S V D K T N G E V T L A G G A
acttccccgcttacaggtggactacctgcgacagcaactgaggatgtgaaaaatgtacaa
  T S P L T G G L P A T A T E D V K N V Q
gttgcaaatgctgatttgacagaggctaaagccgcattgacagcagcaggtgttaccggc
  V A N A D L T E A K A A L T A A G V T G
acagcatctgttgttaagatgtcttatactgataataacggtaaaactattgatggtggt
  T A S V V K M S Y T D N N G K T I D G G
ttagcagttaaggtaggcgatgattactattctgcaactcaaaataaagatggttccata
  L A V K V G D D Y Y S A T Q N K D G S I
agtattgatactacgaaatacactgcagatgacggtacatccaaaactgcactaaacaaa
  S I D T T K Y T A D D G T S K T A L N K
ctgggtggcgcagacggcaaaaccgaagttgtttctattggtggtaaaacttacgctgca
  L G G A D G K T E V V S I G G K T Y A A
agtaaagccgaaggtcacaactttaaagcacagcctgatctggcggaagcggctgctaca
  S K A E G H N F K A Q P D L A E A A A T
accaccgaaaacccgctgcagaaaattgatgctgctttggcacaggttgacacgttacgt
  T T E N P L Q K I D A A L A Q V D T L R
tctgacctgggtgcggtacagaaccgtttcaactccgctattaccaacctgggcaacacc
  S D L G A V Q N R F N S A I T N L G N T
gtaaacaacctgaattctgcccgtagccgtatcgaagattccgactacgcgaccgaagtt
  V N N L N S A R S R I E D S D Y A T E V
tccaacatgtctaaagcgcagattctgcagcaggccggtacctccgttctggcgcaggcg
  S N M S K A Q I L Q Q A G T S V L A Q A
aaccaggttccgcaaaacgtcctctctttactgcgagcagtaccccgggatccgcggctg
  N Q V P Q N V L S L L R A V P R D P R L
caggtcgacgaacaaaaactcatctcagaagaggatctgaatgctgtgggccaggacacg
  Q V D E Q K L I S E E D L N A V G Q D T
caggaggtcatcgtggtgccacactccttgccctttaaggtggtggtgatctcagccatc
  Q E V I V V P H S L P F K V V V I S A I
ctggccctggtggtgctcaccatcatctcccttatcatcctcatcatgctttggcagaag
  L A L V V L T I I S L I I L I M L W Q K
aagccacgttag
  K P R -
METDTLLLWV LLLWVPGSTG DYPYDVPDYA GAQPARSMAQ VINTNSLSLL
TQNNLVKSQS ALGTAIERLS SGLRINSAKD DAAGQAIANR FTANIKGLTQ ASRNANDG
ISIAQTTEGA LNEINNNLQR VRELAVQSAT STNSQSDLDS IQAEITQRLN
EIDRVSGQTQ FNGVKVLAQD NTLTIQVGAN DGETIDIDLK QINSQTLGLD
TLNVQQKYKV SDTAATVTGY ADTTIALDDS TFKASATGLG GTDQKIDGDL
KFDDTTGKYY AKVTVTGGTG KDGYYEVSVD KTNGEVTLAG GATSPLTGGL
PATATEDVKN VQVANADLTE AKAALTAAGV TGTASVVKMS YTDNNGKTID
GGLAVKVGDD YYSATQNKDG SISIDTTKYT ADDGTSKTAL NKLGGADGKT
EVVSIGGKTY AASKAEGHNF KAQPDLAEAA ATTTENPLQK IDAALAQVDT
LRSDLGAVQN RFNSAITNLG NTVNNLNSAR SRIEDSDYAT EVSNMSKAQI
LQQAGTSVLA QANQVPQNVL SLLR AVPRDP RLQVDEQKLI SEEDLNAVGQ
DTQEVIVVPH SLPFKVVVIS AILALVVLTI ISLIILIMLW
QKKPR
```

Underlined regions: Regions encoded by pDisplay
Grey Shaded area: IgK-leader sequence

METDTLLLWV LLLWVPGSTG D

Magenta Shaded area: HA-tag

YPYDVPDYA

Blue Shaded area: fliC

MAQ VINTNSLSLL TQNNLVKSQS ALGTAIERLS SGLRINSAKD
DAAGQAIANR FTANIKGLTQ ASRNANDG ISIAQTTEGA
LNEINNNLQR VRELLAVQSAT STNSQSDLDS IQAEITQRLN
EIDRVSGQTQ FNGVKVLAQD NTLTIQVGAN DGETIDIDLK
QINSQTLGLD TLNVQQKYKV SDTAATVTGY ADTTIALDDS
TFKASATGLG GTDQKIDGDL KFDDTTGKYY AKVTVTGGTG
KDGYYEVSVD KTNGEVTLAG GATSPLTGGL PATATEDVKN
VQVANADLTE AKAALTAAGV TGTASVVKMS YTDNNGKTID
GGLAVKVGDD YYSATQNKDG SISIDTTKYT ADDGTSKTAL
NKLGGADGKT EVVSIGGKTY AASKAEGHNF KAQPDLAEAA
ATTTENPLQK IDAALAQVDT LRSDLGAVQN RFNSAITNLG
NTVNNLNSAR SRIEDSDYAT EVSNMSKAQI LQQAGTSVLA
QANQVPQNVL SLLR

Green Shaded area: PDGFR-transmembrane domain

AVGQ DTQEVIVVPH SLPFKVVVIS AILALVVLTI ISLIILIMLW
QKKPR

Red Shaded residues: altered sequence from flic shown with parentheticals

TQNNL(V)KSQS ALGTAIERLS SGLRINSAKD DAAGQAIANR
FTANIKGLTQ ASRNANDG ISIAQTTEGA LNEINNNLQR
VRELAVQSA(T) STNSQSDLDS IQAEITQRLN EIDRVSGQTQ
FNGVKVLAQD NTLTIQVGAN DGETIDIDLK QINSQTLGLD
TLNVQQKYKV SDTAATVTGY ADTTIALD(D)S TFKASATGLG
GTDQKIDGDL KFDDTTGKYY AKVTVTGGTG KDGYYEVSVD
KTNGEVTLAG GATSPLTGGL PATATEDVKN VQVANADLTE
AKAALTAAGV TGTASVVKMS YTDNNGKTID GGLAVKVGDD
YYSATQNKDG SISI(D)TTKYT ADDGTSKTAL NKLGGADGKT
EVVSIGGKTY AASKAEGHNF KAQPDLAEAA ATTTENPLQK
IDAALAQVDT LRSDLGAVQN RFNSAITNLG NTVNNL(N)SAR
SRIEDSDYAT EVSNMS(K)AQI

Vector maps are shown after the figures
While the invention will be described in relation to certain disclosed embodiments, the skilled person may foresee other embodiments, variations, or combinations which are not specifically mentioned but are nonetheless within the scope of the appended claims.
All references cited herein are hereby incorporated by reference in their entirety.
The expression “comprising” as used herein should be understood to include, but not be limited to, the stated items.
The invention will now be described by way of the following non-limiting examples.

### EXAMPLES

### Example 1

### Flagellin can be Expressed on the Surface of Mammalian Cells

### Cell Culture and Cell Lines

All cell lines were all grown in RPMI 1640 (293FT) or DMEM (HeLa) medium (Life Technologies, Rockville, Md., U.S.A.) with the addition of 5 to 10% heat inactivated Fetal Calf Serum (FCS), 2 mM L-glutamine (Life Technologies, Rockville, Md., U.S.A.), 100 U/ml Penicillin and 100 μg/ml Streptomycin (Life Technologies, Rockville, Md., U.S.A.), 50 μM Betamercaptoethanol (Sigma, St. Louis, Mo., U.S.A.) and 100 mM HEPES (Life Technologies, Rockville, Md., U.S.A.). 293FT cells were obtained from Invitrogen and grown in the aforementioned media with the addition of 500 μg/ml Geneticin (Life Technologies, Rockville, Md., U.S.A.) when not used in experiments. HeLa was obtained from American Type Culture Collection.

Cloning of fliC and Expression Vector Assembly

An overnight culture of Salmonella enterica serovar Typhimurium (pathogenic strain ATCC 14028) was used as a source of genomic DNA to clonefliC (phase-1 flagellin, serotype H i). 50 μl of a liquid overnight culture grown in LB at 37° C. was mixed with 50 μl of TE, heated to 95° C. for 15 min, and centrifuged at high speed in a microcentrifuge. 2 μl of the supernatant was subjected to thermal cycling. PCR was done in the presence of 1 mM dNTPs (Life Technologies, Rockville, Md., U.S.A.), 2 μM MgCl, 1× PCR buffer (Life Technologies, Rockville, Md., U.S.A.), 2 U DNA polymerase (Life Technologies, Rockville, Md., U.S.A.), 20 μM of each primer in a total volume of 50 μl. S. typhimurium DNA was temperature cycled at 96° C. /1 min, 54° C./1 min, 72° C./1.5 min for 30 cycles. flic primer pairs used were chimeric primers containing sequences encoding base-pairs able to be recognized and cut using the DNA restriction enzymes BglII and Smal. Forward primer (fliC 5′-BglII): 5′-GGAAGATCTATGGCACAAGTCATTAATACAAAC-3′, Reverse primer (fliC 3′-SmaI):5′-TCTCCCGGGGTATTAACGCAGTAAAGAGAGGAC-3′. Amplified DNA product was captured using pCR2.1 (Invitrogen, Carlsbad, Calif., U.S.A.) and plasmids containing an insert of the appropriate length were subjected to DNA sequencing. The plasmid containing the capturedfliC ORF was digested with BglII, SmaI and the resulting insert was inserted into the mammalian surface display plasmid pDisplay (Invitrogen, Carlsbad, Calif., U.S.A.) also digested with BgiII and SmaI. The resulting plasmid was subjected to site directed mutagenesis using the QuikChange™ Site-Directed Mutagenesis Kit as described by the manufacturer (Stratagene, La Jolla, Calif., U.S.A.) to eliminate the naturally occurring stop codon (nt 1706-1708) as well as modify residues between the stop codon and those encoded by the pDisplay vector (residues over the junction are fliC-encoded LSLLR]-AVP-[pDisplay-encoded RDPRL]). The resulting plasmid was named pDisp/fliC-Tm. pDisp/fliC-Tm was changed in order to introduce single amino acid (AA) mutations designed to disrupt N-linked glycosylation sites predicted by the NetNGlyc 1.0 Prediction Server (http://www.cbs.dtu.dk/services/NetNGlyc/) at AA 19, 101, 200, 346, 446, and 465. Changes made in thefliC coding region are listed in Table I.

Site directed mutagenesis of thefliC gene in pDisp/fliC-Tm was done leading to the desired AA changes (confirmed by DNA sequencing). The resulting plasmid was named pDisp/fliC-Tm(-gly). Inserts of fliC-Tm and fliC-Tm(-gly) residing on EcoRI/XhoI fragments were excised and inserted into pcDNA3.1/Zeo (+) (Invitrogen, Carlsbad, Calif., U.S.A.) for additional studies. A HindIII fragment containing the open reading frame (ORF) of OVA was removed from pBlueRIP/Ova (a kind gift from C. M. Jones) and was cloned into pcDNA3.1/Zeo(+) (pcDNA3.1/OVA) and is called pOVA.

Cell Transfections, Protein Expression, SDS-PAG and, Western Blotting

Transient transfections in 293FT cells were done using the GenePORTER 2 transfection reagent (Gene Therapy Systems, San Diego, Calif., U.S.A.) according to the manufacturer's instructions. Transient transfections in HeLa cells were done using FuGENE™ 6 (Roche, Indianapolis, Ind., U.S.A.). DNA used for transfection was prepared using a Qiagen EndoFree Plasmid Maxi Kit (Qiagen, Valencia, Calif., U.S.A.). 293FT and HeLa cells used in all in vitro experiments were transfected with 2 μg and 3 μg of DNA, respectively. Two days after transfection, non-adherent cells were removed and adherent cells were harvested by gentle repeated pipetting, washed with PBS, and lysed. Cytoplasmic proteins were isolated by centrifugation and quantitated using the BCA Protein Assay Kit (Pierce Biochemicals, Rockford, Ill., U.S.A.) after which 15 μg of protein was separated on a 10% SDS-polyacrylamide gel and analyzed by Western blotting as described⁵⁰. HA-tagged proteins were detected by using anti-HA tag antibody HA1.1 (at 1:1,000; Covance, Cumberland, Va., U.S.A.) and protein-antibody complexes were visualized using goat anti-mouse IgG antibodies (Pierce Biochemicals, Rockford, Ill., U.S.A.) and the Renaissance Chemiluminescence reagent (NENTM Life Science Products Inc., Boston, Mass., U.S.A.). Proteins were also subjected to Western blotting with polyclonal rabbit antisera (at 1:500) used to clinically detect serotypes of S. Typhimurium (anti-Hi, called here anti-FliC) (State Serum Institute, Copenhagen, Denmark) and protein-antibody complexes were visualized using HRP-conjugated swine anti-rabbit IgG (at 1:1,000; DAKO, Glostrup, Denmark) followed by Enhanced Chemiluminescence detection.

### Cell Surface Expression of FliC

To determine if cells transfected with the expression constructs expressed the FliC-Tm polypeptide at their surface, cells were stained with anti-FliC and anti-HA antibodies followed by FACS® analysis. FliC-Tm expressed at the cell surface of 293FT and HeLa cells was detected using HA1.1 (at 1:100) followed by FITC-conjugated rat. anti-mouse IgG1/κ (at 1:100; PharMingen, San Jose, Calif., U.S.A.) or polyclonal rabbit anti-FliC (at 1:100; State Serum Institute, Copenhagen, Denmark) followed by FITC-conjugated swine anti-rabbit Ig (at 1:100; DAKO, Glostrup, Denmark). Briefly, cells were resuspended, stained 30 min on ice, and washed in PBS containing 1% FCS at 4° C. Cells were stained with secondary antibodies if necessary. Cells were kept on ice until analysis using a four-color FACScan™ flow cytometer (Becton Dickinson Immunocytometry Systems, San Jose, Calif., U.S.A.). Data were processed using the CellQuest program (BD Biosciences, San Jose, Calif., U.S.A.). 293FT cell cultures transfected with either pfliC-Tm or pfliC-Tm(-gly) contained cells detectable with an anti-HA epitope antibody (FIG. 2) but not with an isotype-control antibody (data not shown). Mock transfected cells (data not shown) or cells transfected with an empty vector (FIG. 2) gave background staining. Cells transfected with pfliC-Tm or pfliC-Tm(-gly) also expressed proteins detectable by anti-FliC antibodies (FIG. 2). Similar or greater percentages of cells staining positive for pfliC-Tm or pfliC-Tm(-gly) were detected using the polyclonal anti-FliC antibody. HeLa cells were also transfected with pfliC-Tm, pfliC-Tm(-gly), or empty vector and similar surface expression was observed using anti-HA and anti-FliC antibodies.

### Example 2

### Flagellin Expressing Cells Activates Monocytes

As described above Flagellin can be expressed on the surface of transfected cells. Cells expressing flagellin have been used to activate human monocytes. Monocyte activation

Human PBMC were obtained from non-allergic human volunteers. Peripheral blood was drawn from healthy volunteers and PBMC were isolated from buffy coat preparations by centrifugation on Lymphoprep (Axis-Shield, Oslo, Norway). PBMC were washed three times with PBS using low-speed centrifugation to eliminate thrombocytes and resuspended in RPMI 1640 medium supplemented with 2 mM L-glutamine. 5×10⁶ PBMCs/ml/well.were plated in a 24 well plate (Falcon), then incubated for 2 h at 37° C., 5% CO₂. Non-adherent cells were removed by gentle washing and 1 ml of RPMI 1640 media containing 5% FCS, 100 mM HEPES, 2 mM L-glutamine, Penicillin/Streptomycin, 50 μM Betamercaptoethanol was added to remaining cells and incubated overnight. 293FT or HeLa cells were transfected as indicated above and two days later non-adherent cells were removed and adherent cells were harvested by gentle pipetting, stained with trypan blue and counted. Afterward, adherence-enriched PBMCs (monocytes) were activated with LPS (Sigma, St. Louis, Mo., U.S.A.), recombinant. FliC polypeptide (Alexis Biochemicals, Grünberg, Germany) or mixed with either 5×10⁴ transfected 293FT or transfected HeLa cells and allowed to incubate for 18 h. Total cells were then stained and subjected to flow cytometric analysis. Human monocytes were stained with FITC-conjugated mouse IgG1 anti-human CD80 (at 1:100); PE-conjugated mouse IgG1 anti-CD25 (at 1:100); PerCp-conjugated mouse IgG2a anti-HLA-DR (at 1:100; all from PharMingen, San Jose, Calif., U.S.A.) for 30 min on ice and washed. All cells were stained and analyzed by FACScan™. Monocytes CD80 and CD25 levels studied were gated on HLA-DR positive populations.

ELISAs were carried out on cell culture supernatants and mouse sera. To test for cytokines, supernatants were collected from monocyte cultures after stimulation and frozen at −20° C. Samples were tested in duplicate for the presence of TNFα using a Quantikine® immunoassay according to the manufacturer's instructions (R&D Systems, Minneapolis, Minn., U.S.A.).

Total cultures of transfected cells were washed with PBS then mixed with monocytes, incubated for 18 h, and analyzed for TNFA production and changes in surface expression of CD80 and CD25. Cultures of 293FT cells expressing FliC-Tm or FliC-Tm(-gly) were able to induce monocytes to upregulate cell surface expression of CD80 and CD25 compared to controls (FIG. 3a). The changes induced were similar to those seen after treatrnent with LPS or recombinant FliC polypeptide (FIG. 3c). FliC-Tm or fliC-Tm(-gly) expressing cells were also able to induce production of TNFA (FIG. 3d). Furthermore, supernatants from cultures of transfected 293FT cells were also able to upregulate CD80 and CD25 levels on monocytes (data not shown). NF-κB activation in response to Salmonella-derived FliC (indicative of TLR activation) has been reported to occur in 293 but not in HeLa cells³⁴ raising the possibility that transfection of 293FT cells with FliC-Tm expressing constructs leads to the production of undefined factors from 293FT cells that are able to activate monocytes. To test this hypothesis, transfection and cell mixing experiments were also performed using HeLa cells transfected with pfliC-Tm or pfliC-Tm(-gly). These cells, mixed with monocytes, were also able to induce monocyte activation similar to 293FT expressing FliC-Tm or FliC-Tm(-gly) but not cells transfected with the empty vector (FIGS. 3b, d). Supernatants from cultures of transfected HeLa cells also activated monocytes (data not shown). 293FT and HeLa cells were negative for staining by anti-CD80, anti-CD25 and anti-HLA-DR (data not shown).

### Example 3

### Genetic Vaccination with Flagellin Results in Local Inflammation

C57BL/6J mice were obtained from Charles River (Sulzfeld, Germany) and housed under standard specific pathogen free conditions at the animal facility located at the Swedish Institute for Infectious Disease Control, Stockholm. All procedures were performed under both institutional and national guidelines. Groups of mice, age 6-10 weeks, were used in experiments. Mice were vaccinated using the Helios gene-gun system as described by the manufacturer (BioRad, Hercules, Calif., U.S.A.). Briefly, 0.5 mg of gold particles were coated with 0.5 μg of each plasmid DNA and used to coat the delivery tube. DNA used for vaccination was prepared using a Quiagen EndoFree Plasmid Maxi Kit (Qiagen). Endotoxin/per mg DNA were as follows; pcDNA3.1/OVA (≦5.5×10⁻⁴ EU/μg DNA), pcDNA3.1/Zeo(+) (≦3.625×10⁻⁵ EU/Ig DNA), pcDNA3.1/fliC-Tm (≦2.9×10⁻⁵ EU/Ig DNA), pcDNA3.1/fliC-Tm(-gly) (3.25×10⁻⁵ EU/μg DNA). Endotoxin units were determined using the LAL kit according to the manufacturer's instructions (Bio Whittaker Inc., Walkersville, Md., U.S.A.). We controlled for the binding of DNA to the gold beads by eluting plasmids from bead-coated delivery tubes with TE followed by plasmid transformation, isolation, and DNA restriction enzyme analysis of plasmids isolated from bacterial colonies (data not shown). Abdominal skin of mice was shaved, and the spacer of the gene gun was held directly against the abdomen and the device was discharged at a helium pressure of 500 psi. The site of injection of groups of 6 mice injected with pOvA+pcDNA3.1/Zeo(+), pOVA+pfliC-Tm, or pOVA+pfliC-Tm(-gly) were observed at day 0, 1, 2, 3, and 7. Based on observations of these 3 groups of 6 mice, 3 groups of 7 mice were injected with identical DNA preparations, and one mouse from each group was sacrificed at days 0, and 7. Two mice from each group were sacrificed at days 1, 2, and 3 after injection. Samples isolated from this second series of injected mice were subject to histo-pathological examination. Before biopsies were taken, mice were photographed using a digital camera (4.0 mega-pixels), then skin complete with abdominal wall from the site of injection was harvested. Samples were preserved in neutral-buffered 4% formalin solution overnight followed by immersion in 70% EtOH. Samples were trimmed to include regions adjacent to the injected site, embedded in paraffin, sectioned and stained with hemolysin and eosin (H&E) according to standard protocols. Stained samples were analyzed by light microscopy and photographed at 10, 20, and 40× magnifications. Gross morphology of the injection sites revealed clear differences in the type of responses elicited relative to the type of plasmid delivered (FIG. 4a). Mice injected with the plasmid pOVA+Vector showed a slight local reaction two days post-injection, characterized primarily by a yellowish-brown tinge likely due to deposition of the gold particles. In contrast, mice injected with pOVA+pfliC-Tm or pOVA+pfliC-Tm(-gly) developed severe, but local tissue reactions characterized by swelling and central ulceration of the injection site. Seven days post-injection, the skin was grossly normal in all groups of mice.

Histological analysis of the site of injection, revealed similarities, but also striking differences between mice injected with pOVA+Vector, compared to mice injected with pOVA+pfliC-Tm or pOVA+pfliC-Tm(-gly). In mice sacrificed directly post injection, the distribution of gold particles were found in the epidermis and subepidermal dermis (FIGS. 4b,c,d). On days one and two post injection (FIGS. 4b-d), mice given pOVA+Vector, the developed epidermal hyperplasia, subcomeal pustule formation, increased cellular density in the dermis with infiltration of neutrophillic granulocytes (NG), and an inflammatory reaction extending to, but not involving, the hypodermal fat. On day three, the inflammation was resolving, while the superficial necrotic epidermal layers and pustules, were detaching from the site of injection. In contrast, injection of pOVA combined with either of the FliC-Tm expressing plasmids, led to a more rapid and severe inflammatory reaction, involving also the hypodermis, extending to and involving the superficial part of the panniculus muscle. On day one, epidermal necrosis was observed. in the central injection site and the denuded area covered with fibrin was densely infiltrated by granulocytes. In the dermis and the hypodermis, the inflammatory reaction led to the development of a panniculitis, with dense infiltrates of neutrophilic granulocytes. At day two (FIGS. 4b-d), similar observations were made but in addition there was an increased hyperemia, with marginating neutrophils in the vasculature. At day three, the lateral parts of the injection site displayed epidermal hyperplasia, while the central region still was characterized epidermal pustule formation; the acute inflammatory reaction in the dermal parts persisted, albeit somewhat reduced. The hyperemic vessels were less prominent, and there was evidence of an early wound healing reaction. In all animals tested, there was an aggregation of gold particles in the detaching epidermal region and bead aggregation was seen in the dermis. By day seven (FIG. 4), there was still evidence of epidermal hyperplasia in all groups, with hypergranulosis, and hyperkeratosis, but the inflammation reaction had mostly resolved and many of the remaining gold beads were found clumped together in dermal aggregates. Scar formation was seen in the central injection site, but not in the lateral parts.

### Example 4

### Use of Flagellin as a Genetic Adjuvant Increases Cellular and Humoral Immune Responses

To determine if FliC-Tm expressing vectors could enhance adaptive immune responses to DNA encoded soluble antigen (OVA) we used the gene-gun method to vaccinate mice. Mice were immunized as above with pOVA+Vector, pOVA+pfliC-Tm, or pOVA+fliC-Tm(-gly) according to the immunization schedule illustrated in FIG. 5a. Blood was taken at the indicated days, and serum was tested for the presence of anti-OVA antibodies.

The presence of mouse anti-OVA antibodies was detected as follows. 96 well ELISA plates (Costar assay plate; Costar, Corning, N.Y., U.S.A.) were coated with 10 μg/ml of purified Chicken OVA (Sigma, St. Louis, Mo., U.S.A.) in PBS overnight at 4° C. Plates were washed twice (PBS/0.1% Tween-20), blocked with PBS/1%FCS for 1 h at room-temperature. Serum samples were diluted 1:2 beginning at 1:1,000 for all IgG tests and 1:10 for IgA tests in PBS/1%FCS and added to the OVA-coated plate in duplicate followed by incubation overnight at 4° C. All dilutions were titrated to extinction. Wells were washed three times and either HRP-goat anti-mouse IgG (Fc) (at 1:5,000; Pierce Biochemicals), HRP-rabbit anti-mouse IgG1 (at 1:3,000; Caltag, Burlingame, Calif., U.S.A.), HRP-rabbit anti-mouse IgG2b (at 1:2,000; Caltag, Burlingame, Calif., U.S.A.), HRP-rabbit anti-mouse IgG2c (at 1:4,000; Southern Biotech, Birmingham, Ala., U.S.A.), or HRP-goat anti-mouse IgA (at 1:1,000; Sigma, St. Louis, Mo., U.S.A.) was added to the wells and incubated at room-temperature for 2 h. Wells were washed 5 times and 100 μl of Enhanced K-Blues TMB Substrate (HRP Color-substrate Solution; Neogen Co., Lexington, Ky., U.S.A.) was added. Plates with identical secondary detections were incubated for identical times and substrate reactions were stopped by the addition of 1 M HCl. Plates were analyzed using a Labsystems Genesis ELISA plate reader (Labsystems, Stockholm, Sweden).

Anti-OVA IgG responses were undetectable at day 21 (data not shown). At day 61 anti-OVA IgG levels were measured, and after the final boost, anti-OVA IgG, IgG-isotypes, and IgA were measured (FIGS. 5d, f-i). After one boost, increases in anti-OVA total IgG responses in pfliC-Tm and pfliC-Tm(-gly) vaccinated mice were seen but not in mice given pOVA+Vector (FIG. 5b). After a second boost, higher anti-OVA total IgG titers were observed, including increases in anti-OVA IgG-isotypes IgG1 (FIG. 5f), gG2b (FIG. 5g), and IgG2c (FIG. 5h), as well as IgA (FIG. 5i). Corresponding antibody esponses were slight or undetectable in mice receiving pOVA+Vector alone.

Murine PBMCs were pooled from mice of each group and analyzed 21 days after primary immunization and 31 days after boost one by IFN-γ ELISPOT, essentially as described⁵¹ using a commercial IFN-γ kit (MabTech, Stockholm, Sweden). Antigen restimulation was done in duplicate with PBMCs using the antigens described below. Splenocyte analyses were also made using the commercial IFN-γ ELISPOT system (MabTech, Stockholm, Sweden). Briefly, PBMCs or splenocytes were purified using a ficoll gradient (Amersham Pharmacia Biotech, Piscataway, N.J., U.S.A.) and transferred in triplicates of 200,000 cells/well into 96-well ELISPOT plates (Millipore MAIPN4510). In vitro re-stimulation was done using whole OVA (5 μM, Sigma, St. Louis, Mo., U.S.A.), the H-2Kb OVA derived peptide SIINFEKL (5 μM, Thermo Hybaid, Dreieich, Germany) and the HIV-1 envelope protein rgp 160 (1 μg per well (MicroGeneSys [now Protein Sciences, Meriden, Conn., U.S.A.))) and the H-2Kb immunodominant LCMV peptide GP33 (KAVYNFATM) (5 μM, Thermo Hybaid, Dreieich, Germany). Cell reactivity was confirmed with Concanavalin A. Spot forming cells (SFCs) were quantified 24 h later by an AID ELISPOT reader (Autoimmun Diagnostika, Strassberg, Germany). Statistical analysies were conducted using the student t-test with Excel software.

Lymphocytes were then tested for the presence of antigen-specific T cells in peripheral blood at days 21 and 61, and in the spleens of mice at day 74. ELISPOT analysis of PBMCs at day 21 failed to detect antigen-specific T cell responses in any groups (data not shown). However, analysis of blood at day 61 revealed the presence of circulating IFNγ-producing T cells responding to the H-2Kb-restricted OVA peptide SIINFEKL (residues 257-264) in mice that had received pOVA+pfliC-Tm or pOVA+pfliC-Tm(-gly) but not in mice that had received pOVA+Vector (FIG. 5c). Spleens from mice at day 74 were tested for the presence of IFNγ-producing T cells able to respond to SIINFEKL as well as whole OVA (FIG. 5e). Levels of IFNγ detected in response to SIINFEKL and whole OVA were significantly higher in mice vaccinated with pOVA+pfliC-Tm or pOVA+pfliC-Tm(-gly) than in mice receiving pOVA+Vector alone. T cells from these mice (pOVA+Vector) remained unresponsive to either peptide or polypeptide relative to control peptide or protein.

### REFERENCES

1. Berzofsky, J. A., Ahlers, J. D. & Belyakov, I. M. Strategies for designing and optimizing new generation vaccines. Nat Rev Immunol 1, 209-219 (2001).
2. Calarota, S. et al. Cellular cytotoxic response induced by DNA vaccination in HIV-1-infected patients. Lancet 351, 1320-1325 (1998).
3. MacGregor, R. R. et al. First human trial of a DNA-based vaccine for treatment of human immunodeficiency virus type 1 infection: safety and host response. J Infect Dis 178, 92-100 (1998).
4. Wang, R. et al. Induction of antigen-specific cytotoxic T lymphocytes in humans by a malaria DNA vaccine. Science 282, 476-480 (1998).
5. Kim, J. J. et al. Modulation of antigen-specific humoral responses in rhesus macaques by using cytokine cDNAs as DNA vaccine adjuvants. J Virol 74, 3427-3429 (2000).
6. Kochel, T. J. et al. A dengue virus serotype-1 DNA vaccine induces virus neutralizing antibodies and provides protection from viral challenge in Aotus monkeys. Vaccine 18, 3166-3173 (2000).
7. Raviprakash, K. et al. Dengue virus type 1 DNA vaccine induces protective immune responses in rhesus macaques. J Gen Virol 81, 1659-1667 (2000).
8. Lodmell, D. L., Parnell, M. J., Bailey, J. R., Ewalt, L. C. & Hanlon, C. A. Rabies DNA vaccination of non-human primates: post-exposure studies using gene gun methodology that accelerates induction of neutralizing antibody and enhances neutralizing antibody titers. Vaccine 20, 2221-2228 (2002).
9. Rasmussen, R. A. et al. DNA prime/protein boost vaccine strategy in neonatal macaques against simian human immunodeficiency virus. J Med Primatol 31, 40-60 (2002).
10. Santra, S. et al. Prior vaccination increases the epitopic breadth of the cytotoxic T-lymphocyte response that evolves in rhesus monkeys following a simian-human immunodeficiency virus infection. J Virol 76, 6376-6381 (2002).
11. Timmerman, J. M. et al. Immunogenicity of a plasmid DNA vaccine encoding chimeric idiotype in patients with B-cell lymphoma. Cancer Res 62, 5845-5852 (2002).
12. McConkey, S. J. et al. Enhanced T-cell immunogenicity of plasmid DNA vaccines boosted by recombinant modified vaccinia virus Ankara in humans. Nat Med 9, 729-735 (2003).
13. Raviprakash, K. et al. Needle-free Biojector injection of a dengue virus type 1 DNA vaccine with human immunostimulatory sequences and the GM-CSF gene increases immunogenicity and protection from virus challenge in Aotus monkeys. Virology 315, 345-352 (2003).
14. Subbramanian, R. A. et al. Magnitude and diversity of cytotoxic-T-lymphocyte responses elicited by multiepitope DNA vaccination in rhesus monkeys. J Virol 77, 10113-10118 (2003).
15. Coban, C., Philipp, M. T., Purcell, J. E., Keister, D. B., Okulate, M., Martin, D. S., Kumar, N. Induction of Plasmodium falciparum transmission-blocking antibodies in nonhuman primates by a combination of DNA and protein immunizations. Infection and Immunity 72, 253-259 (2004).
16. Vinner, L. et al. Immunogenicity in Mamu-A*01 rhesus macaques of a CCR5-tropic human immunodeficiency virus type 1 envelope from the primary isolate (Bx08) after synthetic DNA prime and recombinant adenovirus 5 boost. J Gen Virol 84, 203-213 (2003).
17. Scheerlinck, J. Y. Genetic adjuvants for DNA vaccines. Vaccine 19, 2647-2656 (2001).
18. Takeda, K., Kaisho, T. & Akira, S. Toll-like receptors. Annu Rev Immunol 21,335-376 (2003).
19. Cristofaro, P. & Opal, S. M. The Toll-like receptors and their role in septic shock. Expert Opin Ther Targets 7, 603-612 (2003).
20. Hayashi, F. et al. The innate immune response to bacterial flagellin is mediated by Toll-like receptor 5. Nature 410, 1099-1103 (2001).
21. Samatey, F. A. et al. Structure of the bacterial flagellar protofilament and implications for a switch for supercoiling. Nature 410, 331-337. (2001).
22. Bumens, A. P. et al. The flagellin N-methylase gene fliB and an adjacent serovar-specific IS200 element in Salmonella typhimurium. Microbiology 143 (Pt 5), 1539-1547 (1997).
23. Smith, K. D. et al. Toll-like receptor 5 recognizes a conserved site on flagellin required for protofilament formation and bacterial motility. Nat Immunol 4, 1247-1253 (2003).
24. Liaudet, L. et al. Flagellin from gram-negative bacteria is a potent mediator of acute pulmonary inflammation in sepsis. Shock 19, 131-137 (2003).
25. Gewirtz, A. T., Navas, T. A., Lyons, S., Godowski, P. J. & Madara, J. L. Cutting edge: bacterial flagellin activates basolaterally expressed TLR5 to induce epithelial proinflammatory gene expression. J Immunol 167, 1882-1885 (2001).
26. Yu, Y. et al. TLR5-mediated activation of p38 MAPK regulates epithelial IL-8 expression via posttranscriptional mechanism. Am J Physiol Gastrointest Liver Physiol 285, G282-290 (2003).
27. Zeng, H. et al. Flagellin is the major proinflammatory determinant of enteropathogenic Salmonella. J Immunol 171, 3668-3674 (2003).
28. Mizel, S. B., Honko, A. N., Moors, M. A., Smith, P. S. & West, A. P. Induction of macrophage nitric oxide production by Gram-negative flagellin involves signaling via heteromeric Toll-like receptor 5/Toll-like receptor 4 complexes. J Immunol 170, 6217-6223 (2003).
29. Madrazo, D. R., Tranguch, S. L. & Marriott, I. Signaling via Toll-like receptor 5 can initiate inflammatory mediator production by murine osteoblasts. Infect Immun 71, 5418-5421 (2003).
30. McDermott, P. F., Ciacci-Woolwine, F., Snipes, J. A. & Mizel, S. B. High-affinity interaction between gram-negative flagellin and a cell surface polypeptide results in human monocyte activation. Infect Immun 68, 5525-5529. (2000).
31. Means, T. K., Hayashi, F., Smith, K. D., Aderem, A. & Luster, A. D. The Toll-like receptor 5 stimulus bacterial flagellin induces maturation and chemokine production in human dendritic cells. J Immunol 170, 5165-5175 (2003).
32. Agrawal, S. et al. Cutting Edge: different toll-like receptor agonists instruct dendritic cells to induce distinct Th responses via differential modulation of extracellular signal-regulated kinase-mitogen-activated protein kinase and c-Fos. J Immunol 171, 4984-4989 (2003).
33. McSorley, S. J., Ehst, B. D., Yu, Y. & Gewirtz, A. T. Bacterial flagellin is an effective adjuvant for CD4+T cells in vivo. J Immunol 169, 3914-3919 (2002).
34. Keyna, U., Beck-Engeser, G. B., Jongstra, J., Applequist, S. E. & Jack, H. M. Surrogate light chain-dependent selection of Ig heavy chain V regions. J Immunol 155, 5536-5542. (1995).
35. Mashishi, T. & Gray, C. M. The ELISPOT assay: an easily transferable method for measuring cellular responses and identifying T cell epitopes. Clin Chem Lab Med 40, 903-910 (2002).
36. Smith, M. F., Jr. et al. Toll-like receptor (TLR) 2 and TLR5, but not TLR4, are required for Helicobacter pylori-induced NF-kappa B activation and chemokine expression by epithelial cells. J Biol Chem 278, 32552-32560 (2003).
37. Weiss, R. et al. Gene gun bombardment with gold particles displays a particular Th2-promoting signal that over-rules the Thl-inducing effect of immunostimulatory CpG motifs in DNA vaccines. Vaccine 20, 3148-3154 (2002).
38. Morel, P. A., Falkner, D., Plowey, J., Larregina, A. T. & Falo, L. D. DNA immunisation: altering the cellular localisation of expressed protein and the immunisation route allows manipulation of the immune response. Vaccine 22, 447-456 (2004).
39. Krieg, A. M. CpG motifs in bacterial DNA and their immune effects. Annu Rev Immunol 20, 709-760 (2002).
40. Mazzaccaro, R. J. et al. Major histocompatibility class I presentation of soluble antigen facilitated by Mycobacterium tuberculosis infection. Proc Natl Acad Sci U S A 93, 11786-11791 (1996).
41. Simmons, C. P. et al. MHC class I-restricted cytotoxic lymphocyte responses induced by enterotoxin-based mucosal adjuvants. J Immunol 163, 6502-6510 (1999).
42. Cho, H. J. et al. Immunostimulatory DNA-based vaccines induce cytotoxic lymphocyte activity by a T-helper cell-independent mechanism. Nat Biotechnol 18, 509-514 (2000).
43. Hamilton, S. E., Tvinnereim, A. R. & Harty, J. T. Listeria monocytogenes infection overcomes the requirement for CD40 ligand in exogenous antigen presentation to CD8(+) T cells. J Immunol 167, 5603-5609 (2001).
44. Le Bon, A. et al. Cross-priming of CD8+ T cells stimulated by virus-induced type I interferon. Nat Immunol 4, 1009-1015 (2003).
45. Lopez-Boado, Y. S., Espinola, M., Bahr, S. & Belaaouaj, A. Neutrophil serine proteinases cleave bacterial flagellin, abrogating its host response-inducing activity. J Immunol 172, 509-515 (2004).
46. Westerlund-Wikstrom, B. Peptide display on bacterial flagella: principles and applications. Int J Med Microbiol 290, 223-230 (2000).

1-27. (canceled)

28. An isolated flagellin or homologous genes or fragments thereof to improve immunity of a DNA vaccine.

29. Isolated flagellin encoded by a gene according to claim 28 or a fragment thereof to improve immunity against a cellular vaccine.

30. The flagellin according to claim 29, wherein flagellin is expressed as a membrane bound or as a soluble monomer.

31. The flagellin or homologous gene or fragment according to claim 28, where the gene for flagellin has been obtained from any of the following organisms Salmonella, Shigella, Escherichia, Bordetella, Legionella, Burkholderia, Pseudomonas, Helicobacter, Serratia, Bacillus, Vibrio, Caulobacter, Listeria, Clostridium, Borrelia, from other organisms expressing flagellins belonging to group E, from organisms expressing flagelling belonging to group F or any other organisms expressing homologous flagellin genes.

32. The flagellin according to claim 29, where Flagellin is encoded by a nucleic acid construct that encodes for antigens capable of elicitating an immune response.

33. The flagellin according to claim 29, where Flagellin is encoded by a separate construct inoculated in the same localization as the vaccine.

34. The flagellin or homologous gene or fragment according to claim 28, where the vaccine consists of nucleic acids encoding a gene, or protein or a peptide, expressed by a pathogen or a tumour cell.

35. The flagellin or homologous gene or fragment according to claim 28, where the vaccine consists of an attenuated pathogen or tumour cell.

36. The flagellin or homologous gene or fragment according to claim 28, where the vaccine consists of any other substance capable of elicitating specific immunity.

37. A method to induce local inflammation and local toxic effects with the purpose of killing tumor cells or cells involved in autoimmune disease, comprising administering flagellin to a subject in need thereof.

38. The method according to claim 37, where flagellin can be expressed as a membrane bound monomer in the cells or tissue in which inflammation should be induced, whereby induction of local inflammation can result in activation of specific immunity against cells in the tissue, or whereby the specific immunity may result in killing of similar cells at another location, or alternatively the local inflammation may result in local toxicity resulting in killing of cells located in the tissue.

39. The method according to claim 37, where flagellin can be expressed as a soluble monomer.

40. The method according to claim 37, where the gene for flagellin has been obtained from any of the following organisms Salmonella, Shigella, Escherichia, Bordetella, Legionella, Burkholderia, Pseudomonas, Helicobacter, Serratia, Bacillus, Vibrio, Caulobacter, Listeria, Clostridium, Borrelia, from other organisms expressing flagellins belonging to group E, from organisms expressing flagelling belonging to group F or any other organisms expressing homologous flagellin genes.

41. An animal model expressing flagellin in one or more tissues as a model for inflammation.

42. The animal model according to claim 41, where flagellin is expressed under an inducible promoter.

43. The animal model according to claim 41, where the model for inflammation can be used to study the effect of anti-inflammatory drugs.

44. A method for stimulating an immune response, comprising administering the mammalian surface display plasmid pDisplay for the expression of membrane bound flagellin.

45. A plasmid or vector for the expression of membrane bound flagellin with the purpose of stimulating an immune response.

46. A nucleic acid comprising a nucleic acid encoding for at least one antigen capable of elicitating an immune response and a nucleic acid encoding flagellin or a homologous protein or a subfragment of flagellin or a homologous protein having improving effect on immunity in DNA vaccination.

47. The nucleic acid according to claim 46, expressing flagellin as a soluble or membrane bound monomer.

48. The nucleic acid according to claim 46, where the nucleic acid encoding flagellin or a homologous protein or a subfragment thereof has been obtained from any of the following organisms Salmonella, Shigella, Escherichia, Bordetella, Legionella, Burkholderia, Pseudomonas, Helicobacter, Serratia, Bacillus, Vibrio, Caulobacter, Listeria, Clostridium, Borrelia, from other organisms expressing flagellins belonging to group E, from organisms expressing flagelling belonging to group F or any other organisms expressing homologous flagellin genes.

49. The nucleic acid according to claim 46, where the nucleic acid encoding for at least one antigen capable of elicitating an immune response encodes a gene expressed by a pathogen or a tumour cell.

50. A mammalian expression vector comprising a nucleic according to claim 46.

51. A cell transfected with an expression vector according to claim 46.

52. A method for improving the immunogenic response of a DNA vaccination, comprising administering with said vaccine a nucleic according to claim 46.

53. A method for preparing a pharmaceutical composition, comprising adding a flagellin gene, a fragment thereof, flaaellin or a homologue or a fragment thereof, a vector comprising a flagellin gene or a fragment thereof or a cell transfected with a flagellin gene or a fragment thereof or a nucleic acid according to claim 28 to a pharmaceutical composition.

54. A method for improving immunity in DNA vaccination in a mammal wherein a flagellin gene, a fragment thereof, flagellin or a homologue or a fragment thereof, a vector comprising a flagellin gene or a fragment thereof or a cell transfected with a flagellin gene or a fragment thereof or a nucleic acid according to claim 28, is administrated to the mammal in a sufficient amount for effecting immunity improvement.
